(19) United States
(12) Patent Application Publication
(10) Pub. No.: US 2026/0007518 A1
(43) Pub. Date: Jan. 8, 2026

(54) METHODS AND APPARATUS FOR REPAIRING HEART VALVES

(71) Applicant: BRIAN BIANCUCCI, Chelsea, MI (US)

(72) Inventor: BRIAN BIANCUCCI, Chelsea, MI (US)

(21) Appl. No.: 19/136,241

(22) PCT Filed: Jan. 27, 2025
(86) PCT No.: PCT/US2025/013142

§ 371 Date: Jun. 5, 2025

Related U.S. Application Data
(60) Provisional application No. 63/625,938, filed on Jan. 27, 2024.

(51) Int. Cl. A61F 2/24

(52) U.S. Cl. CPC A61F 2/2457; A61F 2/2466

(57) ABSTRACT

A method for treating a patient includes delivering a distal portion of an elongate medical device into a ventricle of a beating closed heart of a subject via a primary skin opening and a secondary puncture, utilizing the distal portion of the elongate medical device to engage a plurality of chordae tendineae extending from a first papillary muscle within the ventricle, at least a first one of the plurality of chordae tendineae attached to a first leaflet of an atrioventricular valve located between the ventricle and an adjacent atrium, and at least a second one of the plurality of chordae tendineae attached to a second leaflet of the atrioventricular valve, approximating the engaged plurality of chordae tendineae to increase an amount of closure possible between the first leaflet and the second leaflet, and locking the plurality of chordae tendineae in their approximated condition.

### BACKGROUND OF THE INVENTION

### Field of the Invention

The field of the invention generally relates to devices and methods of improving functionality of the cardiovascular system of the body, including heart valves, and more particularly the leaflets of heart valves.

### SUMMARY OF THE INVENTION

In one embodiment of the present disclosure, a method for treating a patient includes delivering a distal portion of an elongate medical device into a ventricle of a beating closed heart of a subject via a primary skin opening and a secondary puncture, utilizing the distal portion of the elongate medical device to engage a plurality of chordae tendineae extending from a first papillary muscle within the ventricle, at least a first one of the plurality of chordae tendineae attached to a first leaflet of an atrioventricular valve located between the ventricle and an adjacent atrium, and at least a second one of the plurality of chordae tendineae attached to a second leaflet of the atrioventricular valve, approximating the engaged plurality of chordae tendineae to increase an amount of closure possible between the first leaflet and the second leaflet, and locking the plurality of chordae tendineae in their approximated condition.

In another embodiment of the present disclosure, a method for treating a patient includes non-surgically engaging a plurality of chordae tendineae extending from a first papillary muscle within a ventricle, at least a first one of the plurality of chordae tendineae attached to a first leaflet of an atrioventricular valve located between the ventricle and an adjacent atrium, and at least a second one of the plurality of chordae tendineae attached to a second leaflet of the atrioventricular valve, non-surgically approximating the engaged plurality of chordae tendineae to increase an amount of closure possible between the first leaflet and the second leaflet, and non-surgically locking the plurality of chordae tendineae in their approximated condition.

In yet another embodiment of the present disclosure, a system includes a catheter including an elongate shaft having a distal end and a proximal end and configured to be advanced into a ventricle of a heart of a subject, a grasper carried by the distal end of the shaft and configured to engage at least a first one of a plurality of chordae tendineae extending from a papillary muscle within the ventricle and attached to a first leaflet of an atrioventricular valve located between the ventricle and an adjacent atrium, and to engage at least a second one of the plurality of chordae tendineae attached to a second leaflet of the atrioventricular valve, and to further maintain simultaneous engagement of both of the at least a first one of the plurality of chordae tendineae and the at least a second one of the plurality of chordae tendineae, an actuator carried by the shaft and configured to cause the at least a first one of the plurality of chordae tendineae and the at least a second one of the plurality of chordae tendineae to be moved toward each other, and a fastener carried by the distal end of the shaft and configured to lock the at least a first one of the plurality of chordae tendineae and the at least a second one of the plurality of chordae tendineae with respect to each other.

In still another embodiment of the present disclosure, a system includes a catheter including an elongate shaft having a distal end and a proximal end and configured to be advanced into a ventricle of a heart of a subject, a grasper carried by the distal end of the shaft and configured to engage at least a first one of a plurality of chordae tendineae extending from a papillary muscle within the ventricle and attached to a first leaflet of an atrioventricular valve located between the ventricle and an adjacent atrium, and to engage at least a second one of the plurality of chordae tendineae attached to a second leaflet of the atrioventricular valve, and to further maintain simultaneous engagement of both of the at least a first one of the plurality of chordae tendineae and the at least a second one of the plurality of chordae tendineae, an actuator carried by the shaft and configured to cause the at least a first one of the plurality of chordae tendineae and the at least a second one of the plurality of chordae tendineae to be moved toward each other, and an energy source carried by the distal end of the shaft and configured to deliver energy to fix the at least a first one of the plurality of chordae tendineae and the at least a second one of the plurality of chordae tendineae in place relative to each other.

In yet another embodiment of the present disclosure, a system includes a first catheter including an elongate shaft having a distal end and a proximal end and configured to be advanced into a ventricle of a heart of a subject, a grasper carried by the distal end of the shaft of the first catheter and configured to engage at least a first one of a plurality of chordae tendineae extending from a papillary muscle within the ventricle and attached to a first leaflet of an atrioventricular valve located between the ventricle and an adjacent atrium, and to engage at least a second one of the plurality of chordae tendineae attached to a second leaflet of the atrioventricular valve, and to further maintain simultaneous engagement of both of the at least a first one of the plurality of chordae tendineae and the at least a second one of the plurality of chordae tendineae, an actuator carried by the shaft of the first catheter and configured to cause the at least a first one of the plurality of chordae tendineae and the at least a second one of the plurality of chordae tendineae to be moved toward each other, a second catheter including an elongate shaft having a distal end and a proximal end and configured to be advanced into the ventricle, and a fastener carried by the distal end of the shaft of the second catheter and configured to lock the at least a first one of the plurality of chordae tendineae and the at least a second one of the plurality of chordae tendineae with respect to each other.

In still another embodiment of the present disclosure, a system includes a catheter including an elongate shaft having a distal end and a proximal end and configured to be advanced into a ventricle of a heart of a subject, a grasper carried by the distal end of the shaft of the first catheter and configured to engage at least a first one of a plurality of chordae tendineae extending from a papillary muscle within the ventricle and attached to a first leaflet of an atrioventricular valve located between the ventricle and an adjacent atrium, and to engage at least a second one of the plurality of chordae tendineae attached to a second leaflet of the atrioventricular valve, and to further maintain simultaneous engagement of both of the at least a first one of the plurality of chordae tendineae and the at least a second one of the plurality of chordae tendineae, an actuator carried by the shaft of the first catheter and configured to cause the at least a first one of the plurality of chordae tendineae and the at least a second one of the plurality of chordae tendineae to be moved toward each other, a second catheter including an elongate shaft having a distal end and a proximal end and configured to be advanced into the ventricle, and an energy source carried by the distal end of the shaft of the second catheter and configured to deliver energy to fix the at least a first one of the plurality of chordae tendineae and the at least a second one of the plurality of chordae tendineae in place relative to each other.

### BRIEF DESCRIPTION OF THE DRAWINGS

FIG. 1 is a cross-sectional view of a heart.

FIG. 2 is a perspective view of a mitral valve.

FIG. 3 is a cross-sectional view of a mitral valve in the closing phase of the cardiac cycle, having good leaflet coaptation.

FIG. 4 is a cross-sectional view of a mitral valve in the closing phase of the cardiac cycle, having incomplete leaflet coaptation.

FIG. 5 is a perspective view of a mitral valve showing a first treatment regime, according to an embodiment of the present disclosure.

FIG. 6 is a perspective view of a mitral valve showing a second treatment regime, according to an embodiment of the present disclosure.

FIG. 7 is a perspective view of a mitral valve showing a third treatment regime, according to an embodiment of the present disclosure.

FIG. 8 is a perspective view of a mitral valve being treated by a system for approximating chordae tendineae utilizing a fourth treatment regime, according to an embodiment of the present disclosure.

FIG. 9 is a perspective view of the system for approximating chordae tendineae of FIG. 8 in a first position, according to an embodiment of the present disclosure.

FIG. 10 is a perspective view of the system for approximating chordae tendineae of FIG. 8 in a second position, according to an embodiment of the present disclosure.

FIG. 11 is a perspective view of a system for approximating chordae tendineae in a first position, according to an embodiment of the present disclosure.

FIG. 12 is a perspective view of a system for approximating chordae tendineae in a second position, according to an embodiment of the present disclosure.

FIG. 13 is a perspective view of a system for approximating chordae tendineae in a first position, according to an embodiment of the present disclosure.

FIG. 14 is a perspective view of a system for approximating chordae tendineae in a second position, according to an embodiment of the present disclosure.

FIG. 15 is a perspective view of a system for approximating chordae tendineae in a first position, according to an embodiment of the present disclosure.

FIG. 16 is a perspective view of a system for approximating chordae tendineae in a second position, according to an embodiment of the present disclosure.

FIG. 17 is a perspective view of a system for approximating chordae tendineae in a first position, according to an embodiment of the present disclosure.

FIG. 18 is a perspective view of a system for approximating chordae tendineae in a second position, according to an embodiment of the present disclosure.

FIG. 19 is a perspective view of a system for approximating chordae tendineae in a first position, according to an embodiment of the present disclosure.

FIG. 20 is a perspective view of a system for approximating chordae tendineae in a second position, according to an embodiment of the present disclosure.

FIG. 21 is a perspective view of a ring of a system for approximating chordae tendineae, according to an embodiment of the present disclosure.

FIG. 22 is a perspective view of a ring of a system for approximating chordae tendineae, according to an embodiment of the present disclosure.

FIG. 23 is a perspective view of a system for approximating chordae tendineae, according to an embodiment of the present disclosure.

FIG. 24 is a perspective view of a system for approximating chordae tendineae in a first position, according to an embodiment of the present disclosure.

FIG. 25 is a perspective view of a system for approximating chordae tendineae in a second position, according to an embodiment of the present disclosure.

FIG. 26 is a perspective view of a system for approximating chordae tendineae in a first position, according to an embodiment of the present disclosure.

FIG. 27 is a perspective view of a system for approximating chordae tendineae in a second position, according to an embodiment of the present disclosure.

FIG. 28 is a perspective view of a system for approximating chordae tendineae in a first position, according to an embodiment of the present disclosure.

FIG. 29 is a perspective view of a system for approximating chordae tendineae in a second position, according to an embodiment of the present disclosure.

FIG. 30 is a perspective view of a system for approximating chordae tendineae in a first position, according to an embodiment of the present disclosure.

FIG. 31 is a perspective view of a system for approximating chordae tendineae in a second position, according to an embodiment of the present disclosure.

FIG. 32 is a perspective view of a hook of a system for approximating chordae tendineae, according to an embodiment of the present disclosure.

FIG. 33 is a perspective view of the hook of FIG. 32 loaded into a catheter of a system for approximating chordae tendineae, according to an embodiment of the present disclosure.

FIG. 34 is a perspective view of a system for approximating chordae tendineae in a first position, according to an embodiment of the present disclosure.

FIG. 35 is a perspective view of a system for approximating chordae tendineae in a second position, according to an embodiment of the present disclosure.

FIG. 36 is a perspective view of a system for approximating chordae tendineae, according to an embodiment of the present disclosure.

FIG. 37 is a perspective view of a system for approximating chordae tendineae, according to an embodiment of the present disclosure.

FIG. 38 is a front view representation of the human circulatory system with a system for approximating chordae tendineae inserted from an arterial approach and through the aortic valve to treat a mitral valve, according to an embodiment of the present disclosure.

FIG. 39 is a front view representation of the human circulatory system with a system for approximating chordae tendineae inserted from an arterial approach and through the aortic valve to treat a mitral valve, according to an embodiment of the present disclosure.

FIG. 40 is a front view representation of the human circulatory system with a system for approximating chordae tendineae inserted from a venous approach to treat a tricuspid valve, according to an embodiment of the present disclosure.

FIG. 41 is a front view representation of the human circulatory system with a system for approximating chordae tendineae inserted from a venous approach to treat a tricuspid valve, according to an embodiment of the present disclosure.

FIG. 42 is a front view representation of the human circulatory system with a system for approximating chordae tendineae inserted from a tandem arterial and venous approach to treat a mitral valve, according to an embodiment of the present disclosure.

FIG. 43 is a front view representation of the human circulatory system with a system for approximating chordae tendineae inserted from a venous approach to treat a mitral valve, according to an embodiment of the present disclosure.

### DETAILED DESCRIPTION OF THE ILLUSTRATED EMBODIMENTS

The present disclosure includes methods and devices for reducing or eliminating leakage (regurgitation) through a heart valve. In some diseased hearts, specific pairs or groups of valve chordae are held apart and under tension, which prevents leaflet coaptation and causes regurgitation. Other terms that are used are mitral insufficiency, and mitral incompetence. The present disclosure includes apparatus and methods for approximating particular valve chords (chordae tendineae) that extend to opposing valve leaflets, but which emanate from the same papillary muscle. The approximating process can be accomplished by pulling the chords of opposing leaflets toward each other and then placing and securing a suture loop around them or by placing a substantially rigid structure around them to hold them in close proximity to each other. Approximating the chords can produce approximation of the leaflets of the heart valve, which can reduce or even eliminate regurgitation. Embodiments of systems and methods for accomplishing chordal banding via transcatheter approaches, and other approaches, are described.

Heart disease is a leading cause of death, and diseases of the heart valves are a substantial component of this problem. Mitral valve regurgitation is one type of valve disease which itself is divided into various types based on the cause and the nature of the regurgitation. For example, after a heart attack, weakening of the left ventricular wall causes the posterior wall of the left ventricle and the papillary muscles associated with the left ventricle to move away from the mitral annulus. This results in tensioning of the mitral apparatus and dilation of the annulus. Either or both of these can lead to regurgitation, which exacerbates cardiac dysfunction. This particular disease is known as functional mitral regurgitation, because the valve components (leaflets and chordae) are otherwise intact and functional. The tricuspid valve can similarly be affected by geometric changes of the right ventricle.

Invasive treatments for functional mitral regurgitation commonly focus on structural alterations of the mitral valve in order to restore or approximate the original anatomic geometry. Surgical approaches include implanting an annuloplasty ring to resize the annulus of the valve. Other, less common techniques such as papillary approximation, are used to reduce the inter-papillary distance and transposition of chordae from one leaflet to another. Less invasive approaches have included using external compression of the heart by various apparatus and methods in an attempt to reduce the volume of the ventricle and bring the papillary muscles together. Numerous percutaneous, catheter-based approaches have been described for resizing or reshaping the annulus, reducing the inter-papillary muscle distance or papillary muscle to annulus distance, and/or clipping the valve leaflets together at the point of regurgitation.

Relevant to the present disclosure, the inventor has observed in anatomic studies of the mitral valve that, somewhat counterintuitively, there are chordae emanating from each papillary muscle that connect to both leaflets. In other words, the posteromedial papillary muscle has distinct chordal connections to both the anterior leaflet and the posterior leaflet as does the anterolateral papillary muscle. This chordal arrangement includes the marginal chordae, which attach to the free edge, or margin, of each leaflet. In a diseased valve that becomes tethered, the chordal tension pulls the leaflets out of their normal configuration, keeping the edges of the leaflets from approximating. Thus, full closure of the valve is not possible during the cycle at which closure is needed (e.g., systole). Given the anatomic arrangement noted above, tethering also is also likely to prevent the pair of chordae associated with a given papillary muscle from approximating relative to each other.

Embodiments of the present disclosure enable the approximation and securement of chordae tendineae that attach to opposing heart valve leaflets, but are associated with only one papillary muscle, thus enhancing leaflet closure in a heart valve with impaired closing.

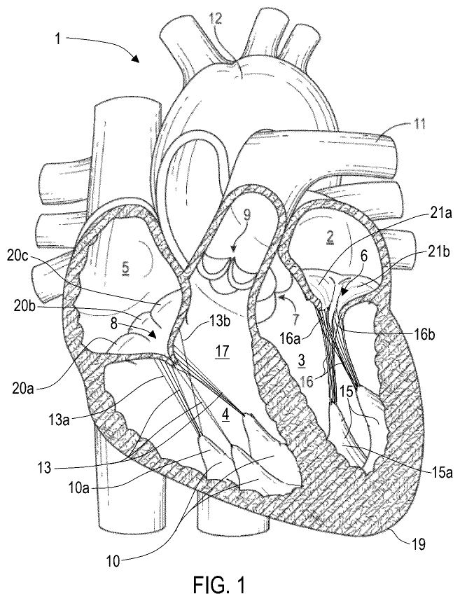

FIG. 1 illustrates an example representation of a heart 1 having various features relevant to certain examples of the present inventive disclosure. The heart 1 includes four chambers, namely the left atrium 2, the left ventricle 3, the right ventricle 4, and the right atrium 5. A wall of muscle 17, referred to as the septum, separates the left 2 and right 5 atria and the left 3 and right 4 ventricles. The inferior tip 19 of the heart 1 is referred to as the apex (or apex region) and is located on the midclavicular line, in the fifth intercostal space.

The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (e.g., systole) and open during ventricular expansion (e.g., diastole). The valves of the heart 1 further include the pulmonary valve 9, which separates the right ventricle 4 from the pulmonary artery 11, and may be configured to open during systole so that blood may be pumped toward the lungs, and close during diastole to prevent blood from leaking back into the heart from the pulmonary artery. The tricuspid valve 8 generally has three cusps/leaflets 20a-c, wherein each one may have a crescent-type shape. The leaflets 20 of the tricuspid valve 8 are commonly referred to as anterior, posterior, and septal. The heart 1 further includes the mitral valve 6, which generally has two cusps/leaflets 21a-b and separates the left atrium 2 from the left ventricle 3. The leaflets 21 of the mitral valve 6 are referred to by varying names: the anterior/aortic/septal leaflet and the posterior/mural leaflet. The mitral valve 6 may generally be configured to open during diastole so that blood in the left atrium 2 can flow into the left ventricle 3, and advantageously close during systole to prevent blood from leaking back from the left ventricle 3 into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant, and press back against the leaflets. As a result, the leaflets/cusps come in apposition to each other, thereby closing the flow passage.

The atrioventricular (e.g., mitral 6 and tricuspid 8) heart valves may further comprise a collection of chordae tendineae 13, 16 and papillary muscles 10, 15 for securing the leaflets of the respective valves to promote and/or facilitate proper coaptation of the valve leaflets and prevent prolapse thereof. The papillary muscles 10, 15, for example, may generally comprise finger-like projections from the ventricle wall. A normal tricuspid valve may comprise three corresponding papillary muscles 10 (two shown in FIG. 1). The valve leaflets 20a-c of the tricuspid valve 8 are connected to the papillary muscles 10 by the chordae tendineae 13, which are disposed in the right ventricle 4 along with the papillary muscles 10. Although tricuspid valves 8 are described herein as comprising three leaflets, it should be understood that tricuspid valves may occur with two or four leaflets in certain patients and/or conditions; the principles relating to chordae tendineae 13, 16 approximation disclosed herein are applicable to atrioventricular valves 6, 8 having any number of leaflets 20, 21 and/or papillary muscles 10, 15 associated therewith.

The right ventricular papillary muscles 10 originate in the right ventricle wall, and attach to the anterior, posterior and septal leaflets 20 of the tricuspid valve 8, respectively, via the chordae tendineae 13. The papillary muscles 10 of the right ventricle 4 may have variable anatomy; the anterior papillary muscle may generally be the most prominent of the papillary muscles 10. The papillary muscles 10 may serve to secure the leaflets 20 of the tricuspid valve 8 to prevent prolapsing of the leaflets 20 into the right atrium 5 during ventricular systole. Tricuspid regurgitation can be the result of papillary dysfunction or chordae rupture.

A normal mitral valve 6 may comprise two papillary muscles 15. In some anatomies, a papillary muscle can comprise a group of two or more muscles. The papillary muscles 15, or muscle groups, originate in the left ventricle wall and project into the left ventricle 3. Generally, the anterior leaflet may cover approximately two-thirds of the valve annulus. Although the anterior leaflet covers a greater portion of the annulus, the posterior leaflet may comprise a larger surface area in certain anatomies.

The valve leaflets 21 of the mitral valve 6 may be prevented from prolapsing into the left atrium 2 by the action of the chordae tendineae 16 connecting the valve leaflets 21 to the papillary muscles 15. The relatively inelastic chordae tendineae 16 are attached at one end to the papillary muscles 15 and at the other to the valve leaflets 21; chordae tendineae 16 from each of the papillary muscles 15 are typically attached to both leaflets of the mitral valve 6. The chordae tendineae 16 keep the leaflets 21 coapting (together) and prevent the valve from opening in the wrong direction, thereby preventing blood to flow back to the left atrium 2. The various chords of the chordae tendineae 16 may have different thicknesses, wherein relatively thinner chords are attached to the free leaflet margin, while relatively thicker chords (e.g., strut chords) are attached farther away from the free margin.

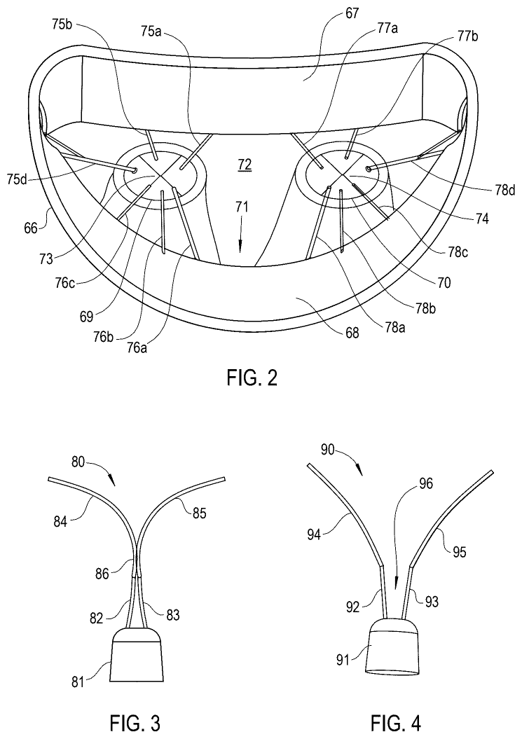

FIG. 2 is a simplified illustration of a mitral valve 66 having an anterior leaflet 67 and a posterior leaflet 68. The mitral valve 66 is shown in a view generally from the left atrium. An anterolateral papillary muscle 69 and a posteromedial papillary muscle 70 extend from a base 71 of the left ventricle 72 to their distal ends 73, 74, which are the initiation for chordae tendineae 75, 76, 77, 78 to extend therefrom. Four types of chordae tendineae 75, 76, 77, 78 are shown. Chordae tendineae 75a-b extend from the anterolateral papillary muscle 69, and connect to the anterior leaflet 67. Chordae tendineae 76a-d extend from the anterolateral papillary muscle 69, and connect to the posterior leaflet 68. Chordae tendineae 77a-b extend from the posteromedial papillary muscle 70, and connect to the anterior leaflet 67. Chordae tendineae 78a-d extend from the posteromedial papillary muscle 70, and connect to the posterior leaflet 68. Thus, each of the papillary muscles 69, 70 have one or more chordae tendineae that connect to the anterior leaflet 67, and one or more chordae tendineae that connect to the posterior leaflet 68. Returning to FIG. 1, papillary muscle 10a includes both chord 13a, which connects to a first leaflet of the tricuspid valve 8, and chord 13b, which connects to a different leaflet of the tricuspid valve 8. Likewise, papillary muscle 15a includes both chord 16a, which connects to a first leaflet of the mitral valve 6, and chord 13b, which connects to a different leaflet of the mitral valve 6.

FIG. 3 illustrates a mitral valve 80 having a papillary muscle 81, a first chord 82 and a second chord 83 extending from the same papillary muscle 81, wherein the first chord 82 connects to an anterior leaflet 84 of the mitral valve 80 and the second chord 83 connects to a posterior leaflet 85 of the mitral valve 80. FIG. 4 illustrates a mitral valve 90 having a papillary muscle 91, a first chord 92 and a second chord 93 extending from the same papillary muscle 91, wherein the first chord 92 connects to an anterior leaflet 94 of the mitral valve 90 and the second chord 93 connects to a posterior leaflet 95 of the mitral valve 90. Both FIG. 3 and FIG. 4 show the mitral valves 80, 90 during closure phase in the cardiac cycle. Mitral valve 80 demonstrates good apposition, with the leaflets 84, 85 having some coaptation, indicated by some overlap 86. Mitral valve 90 demonstrates poor apposition, with an open space 96. In some cases, when especially significant, leaflet prolapse can occur at this space 96. Approximation can be performed on chords on one side only, for example only the anterolateral side, or only the posteromedial side. In other procedures, approximation can be performed on both of these sides, depending on the degree and the location of the leak in the valve. The approximation can include all of the chordae on one side or just some of the chordae on one side. In some cases, the most centrally located chordae are chosen, if those are the chordae that, in that particular patient, attach to a leaflet free edge, and are therefore promising for playing an important part related to valve closure and sealing. The approximation can be done closer to a leaflet edge to enhance the impact on leaflet coaptation. Or, the approximating can be done closer to the papillary tips (top portion); this can potentially lessen the impact on opening (of the valve).

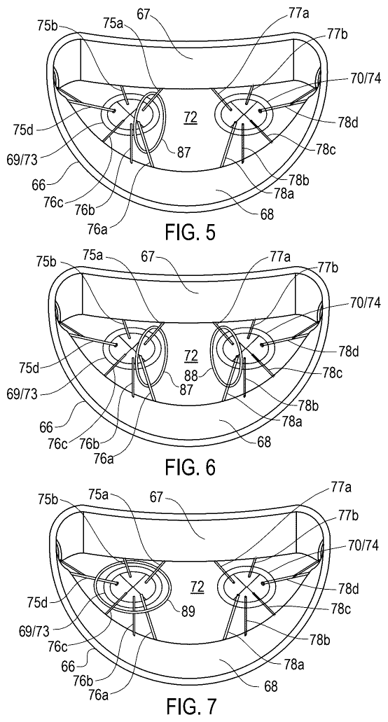

Several embodiments of methods and apparatus for repairing a heart valve utilize approximation (e.g., reducing the distance between two or more chordae tendineae) and then securing the chords in the approximated position. Chords extending from the same papillary muscle, but connecting to different valve leaflets are approximated, in order to increase the amount of apposition and coaptation of two leaflets. The approximation can be performed by a catheter or an elongate probe in order to reach the target area near the valve to be treated. In some cases, when treating a mitral valve, one or more catheters are inserted via a percutaneous puncture of a peripheral artery, or via a cut-down and puncture of the peripheral artery (e.g., femoral, radial) and delivery of the catheter(s) through the descending aorta, aortic arch, ascending aorta, across the aortic valve, and at least partially into the left ventricle. In some cases, when treating a mitral valve, one more catheters are inserted via a percutaneous puncture of a peripheral vein, or via a cut-down and puncture of the peripheral vein (e.g., femoral, subclavian, jugular) and delivery of the catheter(s) through the inferior vena cava and/or the superior vena cava and into the right atrium, and then into the left atrium through a puncture in the septum between the right atrium and the left atrium, and then through the mitral valve at least partially into the left ventricle. In some cases, one or more catheters are inserted via the arterial route as described, while another one or more catheters are inserted via the venous route as described. Embodiments of systems for performing the approximation procedures will be described in more detail. However, FIGS. 5-8 will first be described to show a general approximation and banding of particularly selected chordae tendineae 75, 76, 77, 78, as the end result. Any of these treatment regimes can be performed with devices delivered through the venous approach, as described, the arterial approach, as described, or the combination of these two approaches, as described. In other embodiments, instead of utilizing a percutaneous catheter approach, one or more probes can be placed through the heart wall, for example at the apex of the heart, and into the left ventricle. Following any of the steps for approximating the chordae, the amount of coaptation can be assessed by the clinicians. The assessment can include echocardiography, such as thoracic echocardiography. The determination of regurgitation can be performed utilizing color Doppler ultrasonography.

FIG. 5 illustrates a procedure wherein a band 87 is placed over chord 75a that connects the anterolateral papillary muscle 69 to the anterior leaflet 67, and over chord 76a that connects the anterolateral papillary muscle 69 to the posterior leaflet 68. The chordae 75a, 76a are approximated and locked or set in the approximated condition, to bring the anterior leaflet 67 and the posterior leaflet 68 closer toward preferred apposition.

FIG. 6 illustrates a procedure wherein a first band 87 is placed over chord 75a that connects the anterolateral papillary muscle 69 to the anterior leaflet 67 and a posterior leaflet 68, and over chord 76a that connects the anterolateral papillary muscle 69 to the posterior leaflet 68. The chordae 75a, 76a are approximated and locked or set in the approximated condition, to bring the anterior leaflet 67 and the posterior leaflet 68 closer toward preferred apposition. And, in addition, a second band 88 is placed over chord 77a that connects the posteromedial papillary muscle 70 to the anterior leaflet 67, and over chord 78a that connects the posteromedial papillary muscle 70 to the posterior leaflet 68. The chordae 77a, 78a are approximated and locked or set in the approximated condition, to bring the anterior leaflet 67 and the posterior leaflet 68 even closer toward preferred apposition.

FIG. 7 illustrates a procedure wherein a band 89 is placed over chord 75a and chord 75b that each connect the anterolateral papillary muscle 69 to the anterior leaflet 67, and over chord 76c that connects the anterolateral papillary muscle 69 to the posterior leaflet 68. The chordae 75a, 75b, 76c are approximated and locked or set in the approximated condition, to bring the anterior leaflet 67 and the posterior leaflet 68 closer toward preferred apposition. Bands 87, 88, 89 have a sufficient width or thickness to distribute a securing, but non-damaging force against the chordae 75, 76, for example between about 1 mm and about 20 mm, or between about 2 mm and about 12 mm, or between about 2 mm and about 8 mm, or between about 3 mm and about 6 mm. Multiple band diameters can be provided in a kit, so that a practitioner can choose the best size to approximate the chordae 75, 76 to the right amount. Band diameter can range from about 3 mm to about 15 mm, or between about 4 mm to about 12 mm, or about 5 mm to about 10 mm. The size chosen in a procedure will also depend upon whether only two chords are approximated, or whether significantly more chords are approximated. The determination of regurgitation on color Doppler ultrasonography can aid in the choice of the appropriate band thickness and/or band diameter. A first band size can be placed, and then regurgitation can be assessed via color Doppler. If not acceptable, the band(s) can be removed and another size can be placed, with color Doppler performed again. This can be repeated until the desired result is obtained.

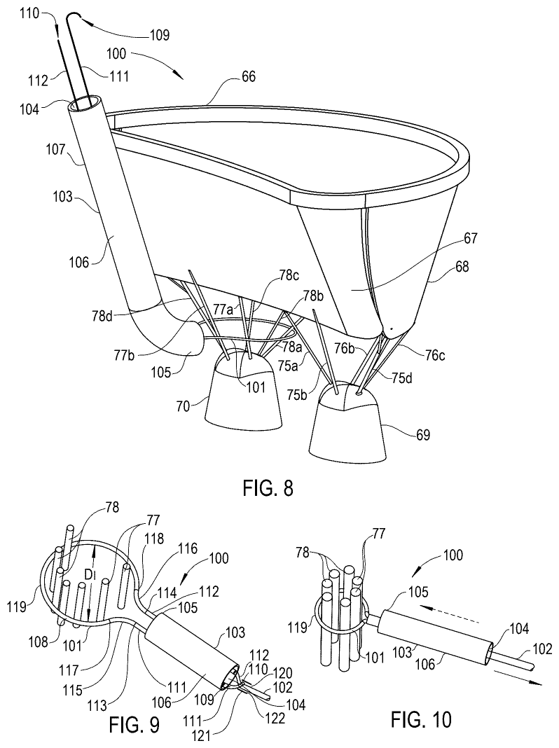

FIG. 8 illustrates a procedure wherein a grasper 101, comprising a lasso-like wire, is placed around chords 77a, 77b that each connect the posteromedial papillary muscle 70 to the anterior leaflet 67, and over chords 78a, 78b, 78c, 78d that connect the posteromedial papillary muscle 70 to the posterior leaflet 68, thus snaring them. The chordae 77a, 77b, 78a, 78b, 78c, 78d are then approximated, to bring the anterior leaflet 67 and the posterior leaflet 68 closer toward preferred apposition. The system for approximation 100 is shown in FIG. 8-10. The system for approximation 100 comprises a delivery catheter 103 having a distal tip 105 a proximal end (not shown), and a lumen 104 extending therethrough. The catheter 103 comprises an elongate shaft 106 configured for placement through the vasculature. The shaft 106 is not shown proximal to a distal portion 107, in order to demonstrate other components inside the lumen 104. The grasper 101 comprises a wire, strand, tether, or other type of line 108 having a first end 109 and a second end 110. A first portion 111 of the line 108 extends from the first end 109 parallel with a second portion 112 of the line 108 that extends from the second end 110. At a distal end 113 of the first portion 111 and at a distal end 114 of the second portion 112, the line 108 transitions to a first laterally-facing concave curve 115 and a second laterally-facing concave curve 116, respectively. At the distal end 117 of the first laterally-facing concave curve 115 and the distal end 118 of the second laterally-facing concave curve 116, the line 108 comprises a continuous turn 119 having a generally circular shape. As shown in FIG. 8, the second end 110 is maintained proximally to a proximal end of the shaft 106, while the first end 109 has a J-shape of fishhook curve 122 (e.g., 100° to 270°, or 150° to) 210° that allows it to be hooked around the desired chordae 77, 78 under fluoroscopic guidance. In some embodiments, at some or all of the line 108 comprises a radiopaque material or a non-radiopaque material having a radiopaque coating or covered with a radiopaque spring coil (e.g., platinum, platinum alloy, gold, tantalum). The user can also inject radiopaque contrast material down the lumen 104 of the catheter 103 in order to better visualize the particular chordae 77, 78. The user can thus determine which chordae 77, 78 are to be selected, which are currently selected, and how the J-shape 122 is moving around them. “Desired chordae” or “desired chordae tendineae,” as used herein, describes chordae tendineae that extend to opposing valve leaflets, but which emanate from the same papillary muscle. In other embodiments, the line 108 comprises a continuous turn 119 having a generally elliptical shape. The lasso can be accomplished by using curved catheter such as a pigtail catheter, or a nitinol tube shaped into a curve, to deliver a wire or suture around the chords. Then, using a snare device, the wire or suture is delivered separately through the same catheter, to capture the free end of the wire or suture and pull it into the catheter. Alternatively, instead of a snare, the capture can be done utilizing a magnet on the end of the wire or suture and then delivering a second wire also with a magnet on the end to connect the two ends and pull the first wire or suture into the catheter.

After the turn 119 of the grasper 101 is placed around the desired chordae 77, 78, the first end 109 and second end 110 of the line 108 are connected to a traction wire 102. In some embodiments, the distal end 120 of the traction wire 102 comprises a crimpable hypo tube 121 having a lumen 122 into which the first end 109 and the second end 110 of the line 108 can be inserted, side-by-side. The hypo tube 121 is then crimped by a hand-held crimping device, to connect the first end 109 and second send 110 to the traction wire 102. In some embodiments, the distal end 120 of the traction wire 102 is configured to snag the first end 109 (e.g., at the J-shape) and hold it snugly. In some embodiments, the distal end 120 of the traction wire 102 is supplied permanently attached to the second end 110 of the line 108. The traction wire 102 is configured to be held in tension by a user (straight arrow, FIG. 10) at a proximal end while the shaft 106 of the catheter 103 is advanced slightly forward (dashed arrow, FIG. 10). Doing so changes the turn 119 of the line 108 from a first condition shown in FIG. 9, to a second condition shown in FIG. 10. In the second condition, the inner diameter D₁ of the turn 119 is reduced, to thus approximating the chords 77, 78 that have been lassoed. The reduction of the inner diameter D₁ of the turn 119 is caused by the inner diameter of the lumen 104 of the shaft 106 forcing down a portion of the turn 119. A constricting band, such as any of the bands 87, 88, 89 of FIGS. 5-7 can be placed onto the approximated chords to lock them into place. The band 87, 88, 89 can be loaded into mechanical jaws at the end of the delivery catheter, or can be secured with a lashing structure. After locking them, the grasper 101 and other components of the system 100 can be retracted and removed from the patient. In some embodiments, the line 108 comprises a single strand. In other embodiments, the line 108 comprises two or more strands. In some embodiments, the line 108 comprises a shape-memory alloy having superelastic/pseudoelastic characteristics, such as nitinol/nickel-titanium alloy. In some embodiments, the line 108 comprises a braided wire or braided filament or fabric material, or suture material. The line 108 can comprise wire or tubing, including shape-memory tubing, or radiopaque drawn filled tube (DFT).

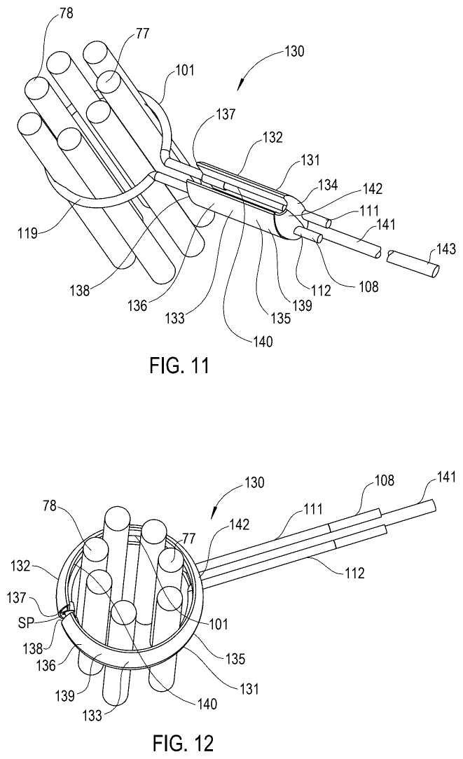

FIGS. 11-12 illustrate a locking system 130 that is configured to be carried by the catheter 103, or by another catheter. In some embodiments, if it is another (second) catheter, the first catheter 103 can be inserted arterially and placed via the aortic arch, across the aortic valve and into the left ventricle, and adjacent to mitral valve, while the second catheter can also be inserted arterially and placed via the aortic arch, across the aortic valve and into the left ventricle, and adjacent to mitral valve. In other embodiments, the first catheter 103 can be inserted arterially and placed via the aortic arch, across the aortic valve and into the left ventricle, and adjacent to mitral valve, while the second catheter can be inserted venously and placed into the left heart (left atrium and/or left ventricle) via a puncture of the septum between the right heart and the left heart. The locking system 130 is configured to lock the constricted grasper 101 in the approximated position, around the “lassoed” or “snagged” chordae tendineae 77, 78. The locking system 130 comprises a constraining band 131 that is folded roughly in half (FIG. 11) and threaded over the first portion 111 of the line 108 and the second portion 112 of the line 108. The constraining band 131 is capable of assuming a ring shape (FIG. 12) as it is pushed simultaneously over two laterally-arrayed substantially semi-circular portions of the turn 119.

The constraining band 131 in its folded configuration, as shown in FIG. 11, comprises a body 135 having a first arm 132 and a second arm 133, each arm 132, 133 substantially linear and configured to project longitudinally. The first arm 132 and second arm 133 are united by a proximal 180° bend 134 or fold. The body 135 comprises an outer surface 136 including a convexity 139 that extends the length of the body 135 between a first end 137 and a second end 138. The convexity 139 can be present, as shown, along the outer-facing portion of the arms 132, 133 and on the outer-facing portion of the bend 134. In other embodiments, the bend 134 does not have the convexity 139, the bend 134 instead comprising a thinned-out portion. The body 135 also comprises an inner channel 140 comprising a concavity, for example, a semi-cylindric channel. The channel 140 is configured to slide freely over the line 108 in a monorail sense, while allowing the body 135 to move from the folded shape of FIG. 11 to the ring shape of FIG. 12, as it is forced over the circular shape of the turn 119 (whether the turn 119 is uncompressed, partially compressed, or fully compressed). An elongate compression mandrel 141 is bonded (adhesive, welding, crimping, brazing, soldering) to a proximal extreme 142 of the body 135 of the constraining band 131, and extends through the lumen of the catheter 103 (or other catheter). A user grasps a proximal end 143 of the compression mandrel 141 to push the mandrel 141 and thus move the body 135 of the constraining band 131 distally, to move it over the line 108 and change its shape from the folded position of FIG. 11 to the ring shape of FIG. 12 that locks the turn 119 in place. The body 135 is configured such that at the full ring shape (FIG. 12) there is at least some space SP between the ends 137, 138, or even substantially zero space. The body 135 in the ring shape stabilizes the approximation of the chordae 77, 78. In some embodiments, the body 135 comprises a ductile metal, such that it is forced by the turn 119 to form the shape of FIG. 12 and maintain it. In other embodiments, the body 135 comprises a shape-memory alloy having memory in the ring shape of FIG. 12 or close to the ring shape of FIG. 12, and is held in a stress-induced Martensite phase in the folded position of FIG. 11, by stresses applied by the first portion 111 of the line 108 and the second portion 112 of the line 108. When moved distally, the body is then allowed to relax and move toward its heat-formed Austenitic shape that is the same as or close to the ring shape of FIG. 12.

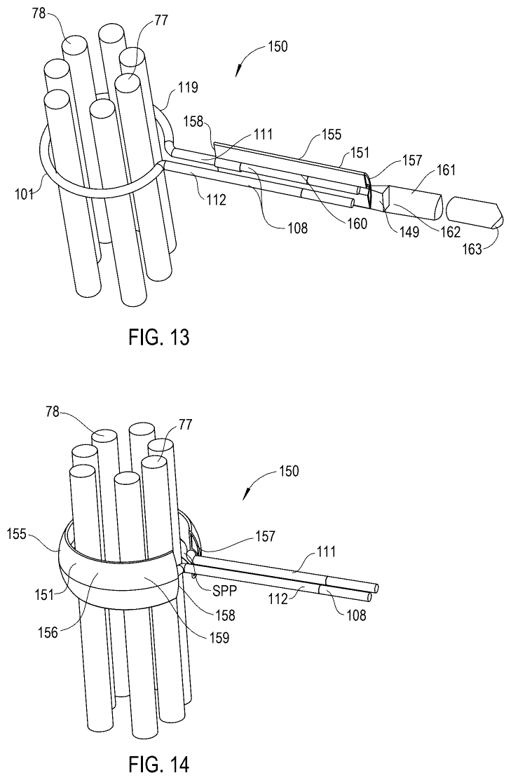

FIGS. 13-14 illustrate a locking system 150 that is configured to be carried by the catheter 103, or by another catheter. In some embodiments, if it is another (second) catheter, the first catheter 103 can be inserted arterially and placed via the aortic arch, across the aortic valve and into the left ventricle, and adjacent to mitral valve, while the second catheter can also inserted arterially and placed via the aortic arch, across the aortic valve and into the left ventricle, and adjacent to mitral valve. In other embodiments, the first catheter 103 can be inserted arterially and placed via the aortic arch, across the aortic valve and into the left ventricle, and adjacent to mitral valve, while the second catheter can be inserted venously and placed into the left heart (left atrium and/or left ventricle) via a puncture of the septum between the right heart and the left heart. The locking system 150 is configured to lock the constricted grasper 101 in the approximated position, around the “lassoed” or “snagged” chordae tendineae 77, 78. The locking system 150 comprises a constraining band 151 that is maintained in a straight condition (FIG. 13) when threaded over the first portion 111 of the line 108. The constraining band 151 is capable of assuming a ring shape (FIG. 14) as it is pushed over the portions of the turn 119.

The constraining band 111 in its straight configuration, as shown in FIG. 13, comprises a body 155 having a first end 157 and a second end 158. The body 155 comprises an outer surface 156 including a convexity 159 that extends the length of the body 155 between the first end 157 and the second end 158. The convexity 159 can be present, as shown, along the outer-facing portion of the body 155 between the two ends 157, 158. The body 155 also comprises an inner channel 160 comprising a concavity, for example, a semi-cylindric channel. The channel 160 is configured to slide freely over the line 108 in a monorail sense, while allowing the body 155 to move from the substantially straight shape of FIG. 13 to the ring shape of FIG. 14, as it is forced over the circular shape of the turn 119 (whether the turn 119 is uncompressed, partially compressed, or fully compressed). An elongate compression mandrel 161 is bonded (adhesive, welding, crimping, brazing, soldering) to the first end 157 of the body 155 of the constraining band 151, and extends through the lumen of the catheter 103 (or other catheter). In some embodiments, the distal end 162 of the mandrel 161 is attached to the first end 157 of the body 155 by a detachable joint 149. The detachable joint 149 can comprise one of a number of detachment systems, including but not limited to electrolytic detachment mechanisms, pressurized detachment, hydraulic detachment mechanisms, mechanical or interlocking detachment mechanisms, heat-activated detachment systems, chemical detachment mechanisms, or frictional detachment systems. Thus, after the body 155 is slid from the position in FIG. 13 to the position in FIG. 14 (straight to circular), the mandrel 161 can be detached from the body 155 by the user, from a control at a proximal location, outside of the catheter, or in the proximal portion of the catheter.

To move the body 155 into position, the user grasps a proximal end 163 of the compression mandrel 161 to push the mandrel 161 and thus move the body 155 of the constraining band 151 distally and around the turn 119, sliding it over the line 108 and changing its shape from the straight position of FIG. 13 to the ring shape of FIG. 14 that locks the turn 119 in place. The body 155 is configured such that at the full ring shape (FIG. 14) there is at least some space SPP between the ends 157, 158, or even substantially zero space. The body 155 in the ring shape stabilizes the approximation of the chordae 77, 78. In some embodiments, the body 155 comprises a ductile metal, such that it is forced by the turn 119 to form the shape of FIG. 14 and maintain it. In other embodiments, the body 155 comprises a shape-memory alloy having memory in the ring shape of FIG. 14 or close to the ring shape of FIG. 14, and is held in a stress-induced Martensite phase in the straight position of FIG. 13, by stresses applied by the first portion 111 of the line 108. When moved distally and around the turn 119, the body is then allowed to relax and move toward its heat-formed Austenitic shape that is the same as or close to the ring shape of FIG. 14.

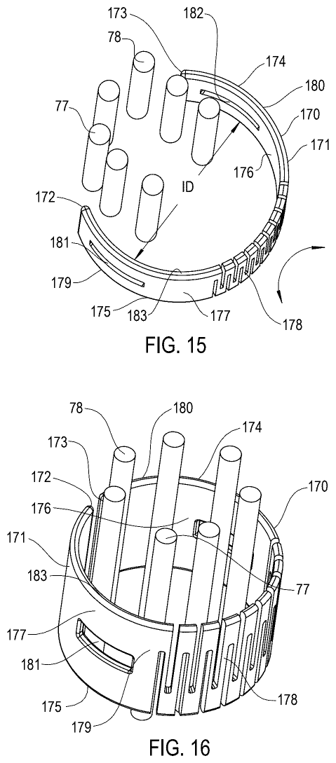

In some embodiments, a band (e.g., bands 87, 88, 89) can be configured with a side/lateral opening or space, and some shape change capability, that allow the band to both grasp/snare and approximate. In some embodiments, the same band can grasp/snare, approximate, and lock, all in one. FIGS. 15-16 illustrate a band 170 in an open configuration (FIG. 15) and a substantially closed configuration (FIG. 16). The band 170 comprises a curved flat band body 171 having a first end 172, a second end 173, a first side 174, a second side, 175, a concave curved inner surface 176, and a convex curved outer surface 177. The band 170 can be configured more than one way. In a first embodiment, the band 170 is configured to be delivered from a distal portion of a catheter by carrying it around the distal outer diameter of the catheter and sliding it off with a longitudinal actuator (e.g., pusher). In a second embodiment, the band 170 is deliverable from a distal portion of an inner lumen of a catheter, by pushing it out of the inner lumen with a longitudinal actuator (e.g., mandrel or guidewire). In some embodiments, the band 170 comprises a polymeric or metallic material and is crimpable by a catheter-delivered crimping device to change the band from the open configuration of FIG. 15 to the substantially closed configuration of FIG. 16. In other embodiments, the band 170 comprises a shape-memory alloy or shape-memory polymer with memory in the closed shape of FIG. 16. In one of these embodiments, the band 170 is heated to a transition temperature, moving from the stress-deformed shape of FIG. 15 to the relaxed shape of FIG. 16. In some cases, this relaxed shape is substantially in a Austenitic phase (if a shape-memory alloy). In another of these embodiments, the band 170 comprises a shape-memory alloy that is in a stress-induced Martensite phase in the open shape of FIG. 15. This can be accomplished, for example, by pulling the first end 172 and second end 173 apart from each other by separation forces, and placing the increased inner diameter ID over the slightly smaller outer diameter of a delivery catheter. Then, as the band is delivered from the delivery catheter (e.g., with a pusher actuator), the band 170 relaxes into the Austenitic state shown in FIG. 16, around the desired chordae tendineae 77, 78.

The band 170 comprises a serpentine shaped circumferential section 178 that is interposed between a first end circumferential section 179 and a second end circumferential section 180. The three sections 179, 178, 180 are continuous and define the outer circumference of the band 170, both in the open shape of FIG. 15 and in the substantially closed shape of FIG. 16. In addition to, or instead of, the shape-memory capabilities or crimpability described, the serpentine shaped circumferential section 178 can be utilized to allow a spring-like opening of the inner diameter ID of the band 170. The band 170 is able to flex at the serpentine shaped circumferential section 178 (curved double-headed arrow) such that the first end 172 and second send 173 can move apart from each other, thus increasing the inner diameter ID of the band 170. Because of the spring action of the serpentine shaped circumferential section 178, the band 170 has mechanical memory to return to the closed shape of FIG. 16. Thus, the band 170 can be delivered in a stretched, increased-ID configuration, as shown in FIG. 15, and then can be delivered off of or out of a catheter and around the desired chordae 77, 78, simultaneously snaring them, and approximating them. In some embodiment, the band 170 is sized appropriately such that the approximation is also sufficiently locking. The spring constant of the serpentine shaped circumferential section 178 can also be adjusted via material choice, thickness, serpentine pattern, such that the approximation is also sufficiently locking. The band includes a first through-slit 181 and a second through-slit 182, each passing through the wall 183 of the band 170. The catheter can include an actuator having jaws, fingers, or hooks that are configured to fit into the slits 181, 182 to manipulate the band 170, moving it off of the catheter and/or pulling it open. The actuator is controlled by control wires that are manipulated from a proximal end of the catheter. The serpentine shaped circumferential section 178 has a square serpentine pattern. In other embodiments, the serpentine pattern is curved, for example, sinusoidal.

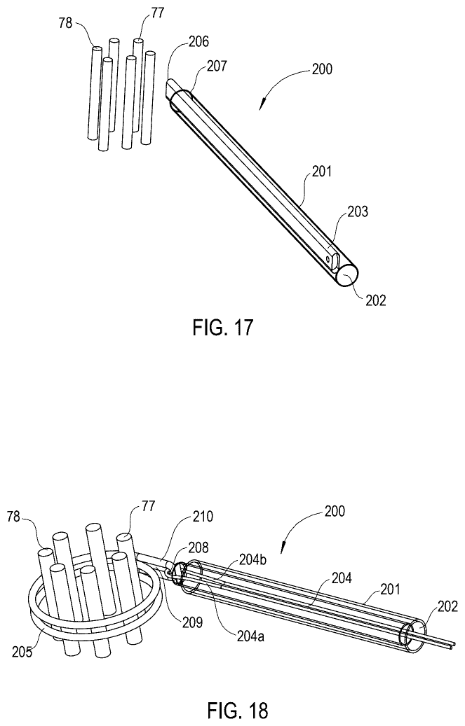

FIGS. 17-18 illustrate a system for grasping 200 comprising a catheter tube 201 having an inner lumen 202 extending therethrough, a pusher mandrel 203, a pull wire/traction wire pair 204 (comprising strand 204a and strand 204b), and a coil grasper 205. The coil grasper 205 has a coiled or spiral shape and comprises a shape-memory alloy, such as nitinol. The grasper 205 comprises a flat wire, or alternatively one, two, or three or more parallel wires, that are heat-set in the spiral or coil shape. A leading end 206 of the grasper 205 exits the distal end 207 of the catheter tube 201 as it is pushed with the pusher mandrel 203 and is able to curve around the desired chordae 77, 78 to snag them. As the grasper 205 continues to exit, the memory-set coil shape coils around the chordae 77, 78 to enclose them completely. In some embodiments, the inner diameter of the grasper 205 is less than the footprint of the chordae 77, 78, and thus, as the grasper 205 coils around the chordae 77, 78, it also causes them to approximate. The distal end 208 of the pull wire(s) 204 is attached to an aperture 209 at a proximal end 210 of the grasper 205. The attachment can be via a loop, a hook, a solder, a weld, a braze, or adhesive or epoxy. Thus, the pull wire 204 can be pulled to pull the grasper 205 back into the lumen 202 at least partially, or fully, if, for example, incorrect chordae 77, 78 were grasped, and an additional attempt at grasping is required. In some embodiments, the coil can be loaded into a catheter lumen in a straightened configuration, for low-profile delivery. It can be pushed out of the distal end of the catheter lumen, causing it to wind around chords as it takes its pre-set coil shape. This device can be combined with a snare or pulling loop.

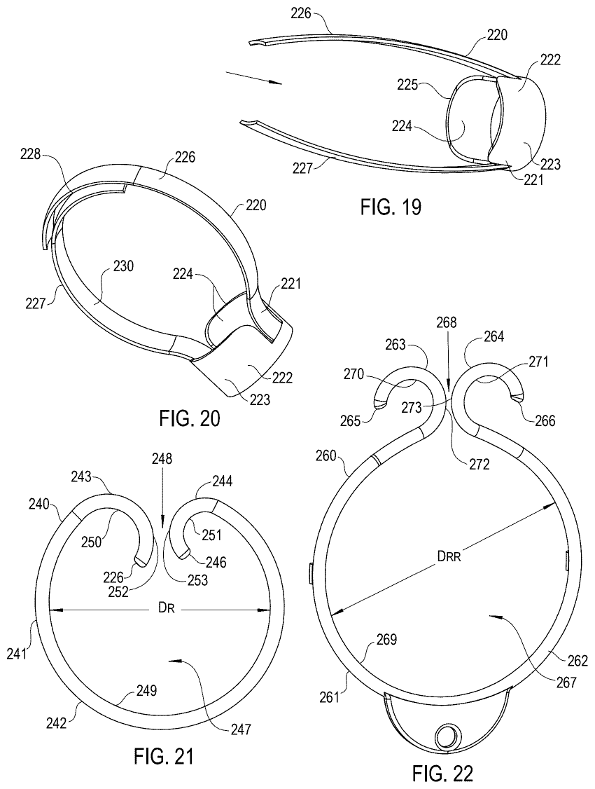

FIGS. 19-20 illustrate a grasper clip 220 that is configured to be delivered from a catheter to grasp desired chordae tendineae 77, 78. In some embodiments, the grasper clip 220 can also serve as an approximator. In some embodiments, the grasper can also serve as a lock. The grasper clip comprises a shape-memory alloy body 221 having a ring base 222 having an outer cylindrical surface 223 and an inner cylindrical surface 224, defining a thin wall 225. The inner cylindrical surface 224 can be carried on an outer diameter of a catheter tube. In some embodiments, the ring base 222 of the body 221 is temporarily crimped onto a catheter tube. Extending distally from the ring base 222 are two arms 226, 227. In a first embodiment, the arms 226, 227 are heat-set in the curved shapes shown in FIG. 20. The curve of arm 226 has a slightly larger radius of curvature than does arm 227. Thus, a small amount of overlap 228 creates a secure, complete circumferential coverage, when wrapped around the chordae tendineae 77, 78. The arms 226, 227 are stress deformed into the substantially longitudinally-extending configurations shown in FIG. 19. Thus, when the gasper clip 220 is carried on the distal end of a catheter, the catheter tip can be delivered up to the desired chordae tendineae 77, 78 to be approximated. The open distal space 229 has then placed over the chordae tendineae 77, 78, and a heating element carried in the catheter is activated to heat the arms 226, 227 causing them to move toward their heat-set shape, as shown in FIG. 20, thereby encircling the chordae tendineae 77, 78. The resulting inner circular face 230 holds the chordae tendineae 77, 78 in place. In some embodiments, the diameter of the inner circular face 230 is smaller than the initial footprint of the chordae tendineae 77, 78, and thus also serves to approximate the chordae tendineae 77, 78. The grasper clip 220 can be detached or displaced from the tip of the catheter shaft, and can thus serve as a lock to maintain the chordae tendineae 77, 78 in the approximated condition. The energy can be delivered from a variety of energy sources, including radio-frequency (RF) or high-intensity focused ultrasound (HIFU). The energy source can be utilized to heat any of the snaring, clipping, approximating, or locking elements described herein, to move them to a heat-set shape (e.g., if exhibiting memory such as shape-memory alloys or shape-memory polymers). The energy source can also be utilized to heat deform or to partially melt any of the snaring, clipping, approximating, or locking elements described herein, to fuse them to the chordae tendineae 77, 78. In some embodiments, the energy source can be utilized to fuse the chordae tendineae 77, 78 directly to each other.

FIG. 21 illustrates a ring 240 of a system for approximating chordae tendineae capable of being delivered onto two or more chordae tendineae 77, 78. The ring 240 has a wire form 241 and a primary curve 242 having a first radius of curvature, and two secondary end curves 243, 244, each having a secondary radius of curvature, less than the primary radius of curvature. The ring 240 further has a first end 245 and a second end 246. The first end 245 and second end 246 each have a blunt end or full-radius for safety. The primary curve 242 and end curves 243, 244 are all outwardly facing convexities. The smaller radius of curvature of each of the end curves 243, 244 causes the ends 245, 246 to point inwardly (invert), within the interior 247 of the ring 240. There is a space 248 between the first end curve 243 and the second end curve 244. In use, the ring 240 is held by an actuator of a catheter and the space 248 is forced over the chordae tendineae 77, 78 to be approximated. The chordae tendineae 77, 78 enter through the space 248 and into the interior 247. When the chordae tendineae 77, 78 enter through the space 248, they encounter no sharp edges. Furthermore, they are guided one-by-one, two-by-two, etc. but then are held in the interior 247 against an inner concave curvature 249 of the primary curve, and are kept from exiting through the space 248 by the two inner concave curvatures 250, 251 of the two secondary end curves 243, 244. Thus, the chordae tendineae 77, 78 are captured and apposed. In some embodiments, the ring 240 can be crimped or otherwise deformed after the desired chordae tendineae 77, 78 enter the interior 247 to push apposing inner surfaces 252, 253 of the two secondary end curves 243, 244 toward each other, minimizing the dimension of the space 248, and even bringing it to zero (causing the facing inner surfaces 252, 253 to touch each other). This additional crimping or deforming causes further apposition, as the inner diameter DR is caused to decrease, causing also the inner area of the ring 240 to decrease. In other embodiments, the ring 240 comprises a shape-memory alloy and is heated to cause the inner surfaces 252, 253 to move toward each other. In some embodiments, the ring 240 is permanently maintained as the lock. Thus, a single ring 240 can be used to perform the snaring, the apposition, and the locking. In other embodiments, the ring 240 comprises a shape-memory alloy in Austenitic state, or another metal or plastic with a sufficient amount of shape retention, that can be reversably deformed with memory recoil. The two inner concave curvatures 250, 251 are gripped by hooks within an actuator in the catheter, to pull them apart to increase a dimension of the space 248. After placing the ring 240 around the desired chordae tendineae 77, 78, the hooks release the inner concave curvatures 250, 251 of the ring 240, leaving it in place. The catheter is then removed from the patient.

FIG. 22 illustrates a ring 260 of a system for approximating chordae tendineae capable of being delivered onto two or more chordae tendineae 77, 78. The ring 260 has a wire form 211 and a primary curve 262 having a first radius of curvature, and two secondary end curves 263, 264, each having a secondary radius of curvature, less than the primary radius of curvature. The ring 260 further has a first end 265 and a second end 266. The first end 265 and second end 266 each have a blunt end or full-radius for safety. The primary curve 262 and end curves 263, 264 are all outwardly facing convexities, but the end curves 263 curve outwardly (evert), oppositely from the primary curve 262. The opposite, outward curvature of each of the end curves 263, 264 causes the end curves 263, 264 to extend outwardly. However, the ends 265, 266 point back inwardly, toward the interior 267 of the ring 240. There is a space 268 between the first end curve 263 and the second end curve 264. In use, the ring 260 is held by an actuator of a catheter and the space 268 is forced over the chordae tendineae 77, 78 to be approximated. The chordae tendineae 77, 78 enter through the space 268 and into the interior 267. When the chordae tendineae 77, 78 enter through the space 268, they encounter no sharp edges. Furthermore, they are guided one-by-one, two-by-two, etc. but then are held in the interior 267 against an inner concave curvature 269 of the primary curve. Thus, the chordae tendineae 77, 78 are captured and apposed. In some embodiments, the ring 260 can be crimped or otherwise deformed after the desired chordae tendineae 77, 78 enter the interior 267 to push apposing inner surfaces 272, 273 of the two secondary end curves 263, 264 toward each other, minimizing the dimension of the space 268, and even bringing it to zero (causing the facing inner surfaces 272, 273 to touch each other). This additional crimping or deforming causes further apposition, as the inner diameter DRR is caused to decrease, causing also the inner area of the ring 260 to decrease. In other embodiments, the ring 260 comprises a shape-memory alloy and is heated to cause the inner surfaces 272, 273 to move toward each other. In some embodiments, the ring 260 is permanently maintained as the lock. Thus, a single ring 260 can be used to perform the snaring, the apposition, and the locking. In other embodiments, the ring 260 comprises a shape-memory alloy in Austenitic state, or another metal or plastic with a sufficient amount of shape retention, that can be reversably displaced with memory recoil. Two inner concave curvatures 270, 271 of the secondary end curves 263, 264 are gripped by hooks within an actuator in the catheter, to pull them apart to increase a dimension of the space 268. After placing the ring 260 around the desired chordae tendineae 77, 78, the hooks release the inner concave curvatures 270, 271 of the ring 260, leaving it in place. The catheter is then removed from the patient.

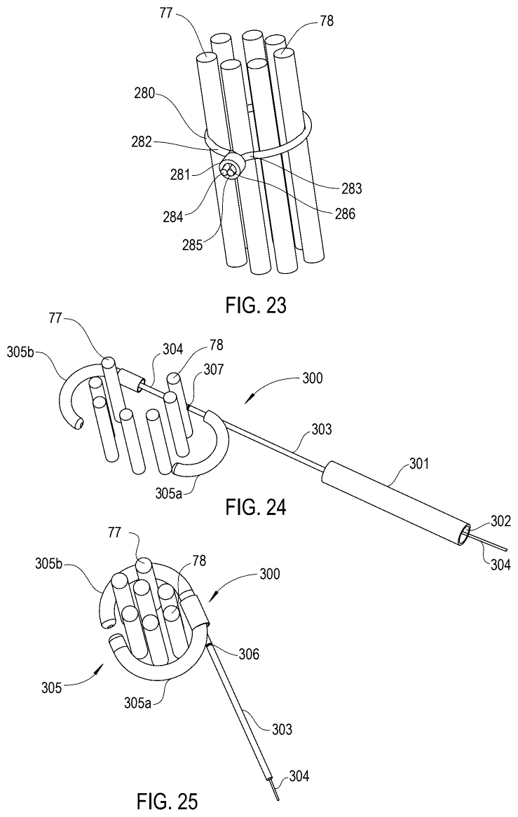

FIG. 23 illustrates a snare 280 comprising suture material or monofilament that is hooked around the desired chordae tendineae 77, 78. A locking collar 281 is slid by an actuator of a catheter over the two extending filament arms 282, 283 of the hooked snare 280. The locking collar 281 has an inner lumen 286. The locking collar 281 is pushed tight while traction on the filament arms 282, 283 is maintained, thus approximating the chosen desired chordae tendineae 77, 78 by the decreased inner diameter of the encircling filament. Then the locking collar 281 is crimped onto the filament arms 282, 283 to lock the approximation in place. In other embodiments, the locking collar 281 is deformed by heating (energy source such as HIFU or RF) to fuse it to the filament arms 282, 283 and lock in the approximation. The filament arms 282, 283 can then be severed by a cutter carried on a catheter. The cutter can comprise an electrified wire element. Alternatively, the filament arms 282, 283 can be detachable (electrolytic detachment mechanisms, pressurized detachment, hydraulic detachment mechanisms, mechanical or interlocking detachment mechanisms, heat-activated detachment systems, chemical detachment mechanisms, or frictional detachment systems). The remaining ends 284, 285 can thus extend a small amount or not at all out of the locking collar 281. In some embodiments, the filament arms 282, 283 can be cinched. In some embodiments, the filament arms can include a zip tie-like structure, that allows incremental tightening and locking.

FIGS. 24-25 illustrate a system for grasping 300 comprising a catheter tube 301 having an inner lumen 302 extending therethrough, a tubular shaft 303, a pull wire/traction wire 304, and a grasper 305. The grasper 305 comprises a proximal curved jaw 305a that is rigidly attached to the tubular shaft 303 and a distal curved jaw 305b that is rigidly attached to the pull wire/traction wire 304. The curved jaws 305a, 305b can comprise a superelastic shape-memory alloy element that is capable of being compressed into a straight shape for delivery through a catheter lumen 302, but expands to the curved shape when delivered from the catheter lumen 302. As shown in FIG. 24, the grasper 305 is moved in an open state and the curved jaws 305a, 305b are placed around the chordae tendineae 77, 78 to be grasped. A user then maintains traction on the traction wire 304 and advances the tubular shaft 303 to cause the curved jaws 305a, 305b to close around the chordae tendineae 77, 78. In some embodiments, the tubular shaft 303 is longitudinally displaceable within the lumen 302 of the catheter tube 301. In other embodiments, the tubular shaft 303 is statically bonded within the lumen 302 of the catheter tube 301, so that the user manipulates the catheter tube 301 to thereby manipulate the tubular shaft 303. In some techniques, the user can push the tubular shaft 303 distally while simultaneously pulling the traction wire 304 proximally, to close the grasper over the chordae 77, 78. In some techniques, the user can maintain the position of the of the tubular shaft 303 and pull the pull wire/traction wire 304. In some techniques, as described, the user can maintain the position of the traction wire 304 and push the tubular shaft 303. In some embodiments, the inner area of the closed grasper 305 is less than the footprint of the chordae 77, 78, and thus, as the grasper 305 closes around the chordae 77, 78, it also causes them to approximate. In some embodiments, the tubular shaft 303 can be coupled to the proximal curved jaw 305a by a first detachable joint 306 and the pull wire/traction wire 304 can be coupled to the distal curved jaw 305b by a second detachable joint 307. When the jaws 305a, 305b are closed around the chordae 77, 78, the two detachable joints 306, 307 are detached together (electrolytic detachment mechanisms, pressurized detachment, hydraulic detachment mechanisms, mechanical or interlocking detachment mechanisms, heat-activated detachment systems, chemical detachment mechanisms, or frictional detachment systems), fusing the pull wire/traction wire 304 to the distalmost portion of the tubular shaft 303 and detaching the remainder of the tubular shaft 303 and the proximally-extending portion of the pull wire/traction wire 304. Thus, in addition to the snaring and approximating capabilities of the grasper 305, the grasper 305 can also be utilized as a lock. In some embodiments, an internal snap between an external portion of the pull wire/traction wire 304 and the internal diameter of the tubular shaft can be configured and located such that when the jaws 305a, 305b close, a locking occurs between the jaws 305a, 305b.

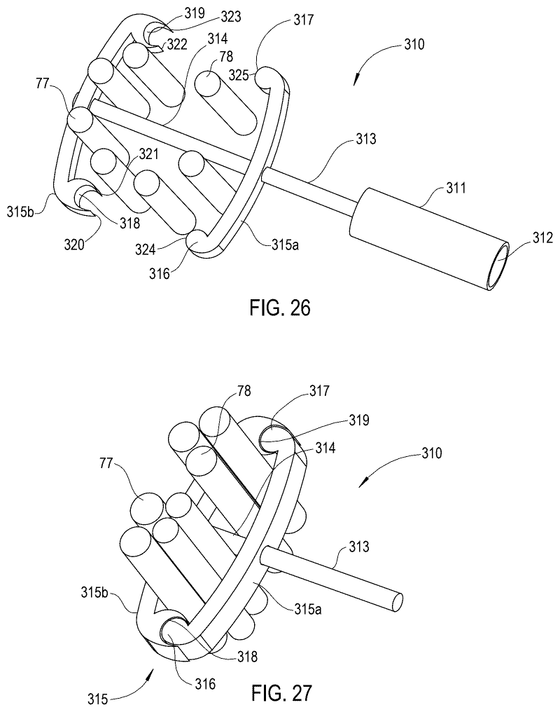

FIGS. 26-27 illustrate a system for grasping 310 comprising a catheter tube 311 having an inner lumen 312 extending therethrough, a tubular shaft 313, a pull wire/traction wire 314, and a grasper 315. The grasper 315 comprises a proximal curved jaw 315a that is rigidly attached to the tubular shaft 313 and a distal curved jaw 315b that is rigidly attached to the pull wire/traction wire 314. The curved jaws 315a, 315b can comprise a superelastic shape-memory alloy element that is capable of being compressed into a straight shape for delivery through a catheter lumen 312, but expands to the curved shape when delivered from the catheter lumen 312. The curvature of the jaws 315a, 315b is slight, creating an oval-shaped inner area for the chordae tendineae 77, 78 to be contained. As shown in FIG. 26, the grasper 315 is moved in an open state and the jaws 315a, 315b are placed around the chordae tendineae 77, 78 to be grasped. A user then maintains traction on the traction wire 314 and advances the tubular shaft 313 to cause the jaws 315a, 315b to close around the chordae tendineae 77, 78. In some embodiments, the tubular shaft 313 is longitudinally displaceable within the lumen 312 of the catheter tube 311. In other embodiments, the tubular shaft 313 is statically bonded within the lumen 312 of the catheter tube 311, so that the user manipulates the catheter tube 311 to thereby manipulate the tubular shaft 313. In some techniques, the user can push the tubular shaft 313 distally while simultaneously pulling the traction wire 314 proximally, to close the grasper over the chordae 77, 78. In some techniques, the user can maintain the position of the of the tubular shaft 313 and pull the pull wire/traction wire 314. In some techniques, as described, the user can maintain the position of the traction wire 314 and push the tubular shaft 313. In some embodiments, the inner area of the closed grasper 315 is less than the footprint of the chordae 77, 78, and thus, as the grasper 315 closes around the chordae 77, 78, it also causes them to approximate. In some embodiments, the tubular shaft 313 can be coupled to the proximal jaw 315a by a first detachable joint (not shown) and the pull wire/traction wire 314 can be coupled to the distal jaw 315b by a second detachable joint (not shown), as described in relation to the embodiment of FIGS. 24-25. Thus, in addition to the snaring and approximating capabilities of the grasper 315, the grasper 315 can also be utilized as a lock.

In some embodiments, an internal snap between an external portion of the pull wire/traction wire 314 and the internal diameter of the tubular shaft can be configured and located such that when the jaws 315a, 315b close, a locking occurs between the jaws 315a, 315b. However, this is not required, as the jaws 315a, 315b have an additional locking capability. The proximal jaw 315a includes distally-extending snap tabs 316, 317, and the distal jaw 315b includes proximally-open snap sockets 318, 319. The socket 318 is defined by bookend socket arms 320, 321 and the socket 319 is defined by bookend socket arms 322, 323. The tab 316 is configured to snap into the socket 318. The tab 317 is configured to snap into the socket 319. With the tabs 316, 317 engaged within the sockets 318, 319, the jaws 315a, 315b are thus locked together, and the chordae 77, 78 are locked together in their approximated condition. The jaws 315a, 315b comprise a rigid polymer or a metallic material with some flexibility at the snap arms 320, 321, 322, 323 such that the temporary deformation during snapping occurs mainly at the snap arms 320, 321, 322, 323. The snap tabs 316, 317 each have a radiused leading edge 324, 325 that each the entry into the sockets 318, 319, respectively.

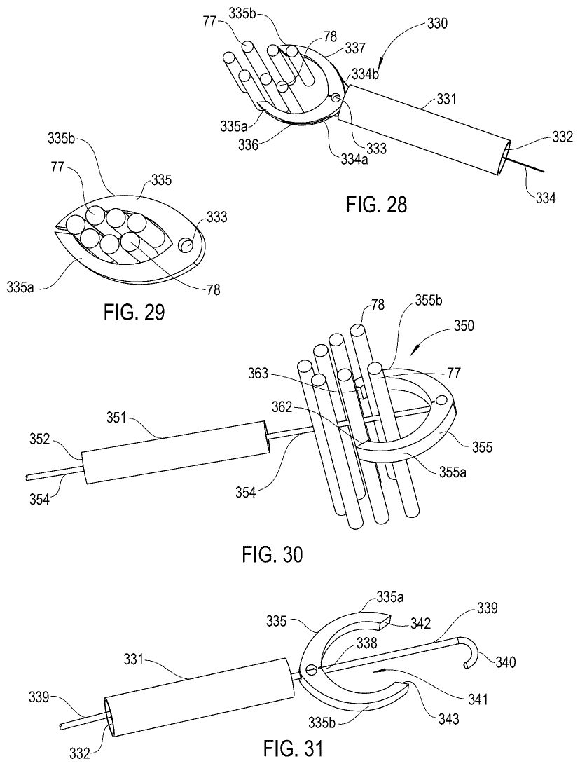

FIGS. 28-29 illustrate a system for grasping 330 comprising a catheter tube 331 having an inner lumen 332 extending therethrough, pull wire/traction wire 334, and a grasper 335. The grasper 335 comprises a monolithic superelastic shape-memory alloy featuring a first curved jaw 335a and a second curved jaw 335b arrayed laterally in relation to each other. The curved jaws 335a, 335b are heat set in a compressed shape shown in FIG. 29. Once delivered though the inner lumen 332 of the catheter tube 331, the jaws 335a, 335b are opened and placed around the desired chordae tendineae 77, 78 for approximation The curvature of the jaws 315a, 315b is such that it creates an eye-shaped inner area for the chordae tendineae 77, 78 to be contained. The pull wire/traction wire 334 bifurcates distally into a first strand 334a that is connected to a lateral portion 336 of the jaw 315a and a second strand 334b that is connected to a lateral portion 337 of the jaw 315b. As shown in FIG. 28, the grasper 335 is moved in an open state by pulling the pull wire/traction wire 334 within the catheter tube 331, causing the strands 334a, 334b to force open the jaws 335a, 335b. The jaws 335a, 335b, in the open position of FIG. 28, are then placed around the chordae tendineae 77, 78 to be grasped. A user then releases the traction or tension on the traction wire 334 to allow the jaws 335a, 335b to close around the chordae tendineae 77, 78. In some embodiments, the inner area of the closed grasper 335 is less than the footprint of the chordae 77, 78, and thus, as the grasper 335 closes around the chordae 77, 78, it also causes them to approximate. In some embodiments, the strands 334a, 334b or the pull wire/traction wire 334 can be coupled to the grasper 335 by a first detachable joint (not shown), as described in relation to the embodiment of FIGS. 24-25. Thus, in addition to the snaring and approximating capabilities of the grasper 335, the grasper 335 can also be utilized as a lock. The grasper 335 comprises an aperture 333 passing through a central, proximal portion. This removal of material adds flexibility or pliability. In some embodiments, the grasper 335 can have a general V-shape and in other embodiments, the grasper 335 can have a general C-shape. In other embodiments, two separate jaws can be used, with the aperture 333 replaced by a pivot joint or a hinge. In some embodiments, the hinge can be passive (e.g., no spring or biasing member). In other embodiments, there is a spring to make the jaws biased toward a closed position. In still other embodiments, one or both of the jaw tips can be magnetized or comprise a magnet or a magnetic material. The magnetic attraction produces an effect similar to a spring bias, by magnetically biasing the jaws toward closure.

FIG. 31 illustrates an over-the-wire embodiment of the grasper 335 of FIGS. 28-29 incorporating a longitudinal passageway 338. A hook 339 is slidable within the longitudinal passageway 338 of the grasper 335. The hook 339 is an elongate device having a proximal end that can be manipulated by a used to pull and push the distal hooked end 340. The distal hooked end can comprise an arc of between 160° and 250°, or between 170° and 200°, or between 180° and 190°. The hook 339 is advanced through the chordae tendineae 77, 78 and hooked around the desired chordae tendineae. The hook 339 can be easily manipulated to grasp a first desired chord, and then hook additional desired chords. The user then maintains support via the catheter tube 331 and pulls the grasped chordae 77, 78 into the opening 341 (“mouth”) of the grasper 335, between the jaws 335a, 335b. The grasper 335 includes lead in tapers 342, 343 to that, in a first embodiment, the grasper 335 is heat-set to be in the closed position of FIG. 29, but the grasped chordae 77, 78 temporarily force the jaws 335a, 335b open, while the hook 339 is pulling the chordae 77, 78 into the opening 341. In other embodiments, the jaws 335a, 335b can be held open (e.g., via the strands 334a, 334b, as in FIG. 28) as the chordae 77, 78 are pulled by the hook 339 into the opening 341, and then released after the desired chordae 77, 78 are within the opening 341. In other embodiments, the grasper 335 can be heat-set in the closed position of FIG. 28, but deformed into an open position (as in FIG. 28). After pulling the chordae 77, 78 the opening 341 with the hook 339, a catheter-carried energy source (e.g., HIFU, RF) is utilized to heat the grasper 335 to thereby move the jaws 335a, 335b into the closed position of FIG. 29, locking around the chordae tendineae 77, 78 in their approximated condition.

FIG. 30 illustrates yet another embodiment of a system for grasping 350 comprising a catheter tube 351 having an inner lumen 352 extending therethrough, a manipulation wire 354, and a grasper 355. The grasper 355 comprises a monolithic superelastic shape-memory alloy featuring a first curved jaw 355a and a second curved jaw 355b arrayed laterally in relation to each other. The curved jaws 355a, 355b are heat set in a compressed shape. Once delivered though the inner lumen 352 of the catheter tube 351, the manipulation wire 354 is extended to move the grasper 355 past the chordae 77, 78. Then the desired chordae 77, 78 are selected and the manipulation wire 354 is pulled and manipulated such that the jaws 355a, 355b are opened (via the lead ins 362, 363) and the desired chordae tendineae 77, 78 are pulled inside the grasper 355 and are approximate. The curvature of the jaws 355a, 355b is such that it creates an eye-shaped inner area for the chordae tendineae 77, 78 to be contained. The manipulation wire 354 can be coupled to the grasper 355 by a first detachable joint (not shown), as described in relation to the embodiment of FIGS. 24-25. Thus, in addition to the snaring and approximating capabilities of the grasper 355, the grasper 355 can also be utilized as a lock.

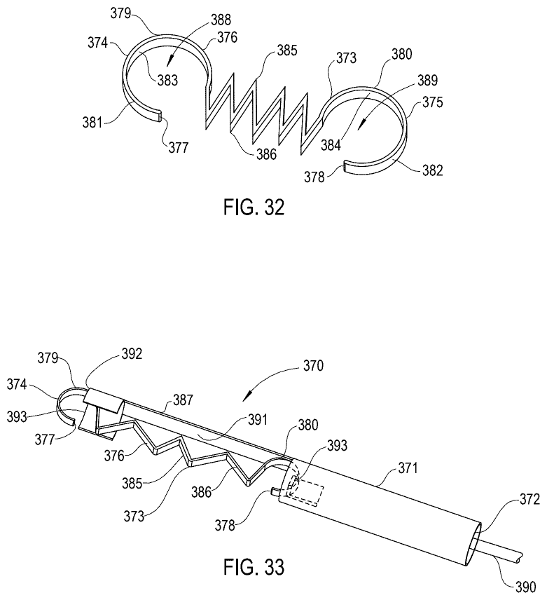

FIGS. 32-33 illustrate a system for grasping 370 comprising a catheter 371 having an inner lumen 372 extending therethrough, and a spring-loaded double hook 373 for engaging and approximating two or more chordae tendineae 77, 78. The double hook 373 comprises a first end 374 and a second end 375. The double hook 373 is configured to be loaded onto the catheter 371 in an expanded, spring-biased position, as shown in FIG. 33, wherein the first end 374 is distally oriented and the second end 375 is proximally oriented. The double hook 373 is formed from a metallic flat wire or a flat semi-rigid, shaped plastic ribbon 376. The wire or ribbon can include radiopaque material (e.g., platinum) to be visible on fluoroscopy, or can have a radiopaque coating. In other embodiments, the double hook 373 is formed from round wire or ribbon, or other cross-sectional shapes. The wire 376 has a first end 377 and a second end 378. The double hook 373 has a shape comprising three general sections, distal section 379 and proximal section 380 that each comprise a hook portion 381, 382 having an internally curved face 383, 384 that is configured to grasp one or more chordae tendineae 77, 78. In between the distal section 379 and the proximal section 380 is a connecting spring section 385 having a zig-zag pattern 386. FIG. 32 shows the zig-zag pattern 386 with the spring section 385 in its relaxed state. The spring section 385 allows a certain amount of elastic deformation upon being longitudinally stretched and elongated. The amount of stretch and elongation shown in the configuration of FIG. 33, attached at the distal portion 387 of the catheter 371 is within the elastic limit of the material and the zig-zag pattern 386. A stop 392 located at a distal end 393 of the catheter 371 and flexible hook 393, proximal to a pocket section 391 maintain the double hook 373 in the stretched state, and in place in the pocket 391 of the catheter 371. When released from the longitudinally elongated position of FIG. 33, the zig-zag pattern will return to the related state shown in FIG. 32. When loaded onto the catheter 371 in the elongated position shown in FIG. 33, the double hook 373 can be delivered by the catheter (e.g., over a guidewire) to a desired location within the heart. The double hook 373 is positioned such that the internally curved faces 383, 384 are placed over desired chordae tendineae 77, 78 to load the chordae tendineae 77, 78 within the circular openings 388, 389. For example, in some cases, chordae tendineae 77 attached to a first leaflet of a heart valve are grasped by the first circular opening 388 and chordae tendineae 78 attached to a second leaflet of the heart valve are grasped by the second circular opening 389. A butting mandrel 390 extends down the lumen 372 and can be pushed forward by a user to detach the stretched double hook 373 from the distal pocket 391 in the catheter 371 by disrupting the flexible hook 393 and/or disrupting the engagement of the distal section 379 within the stop 392. This frees the double hook 373 and allows it to shorten, via the memory-controlled shortening of the spring section 385 (from the stretched state of FIG. 33 toward the relaxed state of FIG. 32). Thus, the chordae tendineae 77, 78 are approximated by the movement of the hook portions 381, 382 toward one another. The double hook 373 thus locks the chordae tendineae 77, 78. Further securement is possible, by advancing a catheter with energy delivery capabilities (HIFU, RF) to heat the double hook 373 and/or the chordae tendineae 77, 78, to fuse them together permanently. In alternative embodiments, instead of facing the same direction (as in the embodiment of FIGS. 32-33), internally curved faces 383, 384 of the hook portions 381, 382 can face opposite directions, thus giving the double hook 373 more of an S-shape.

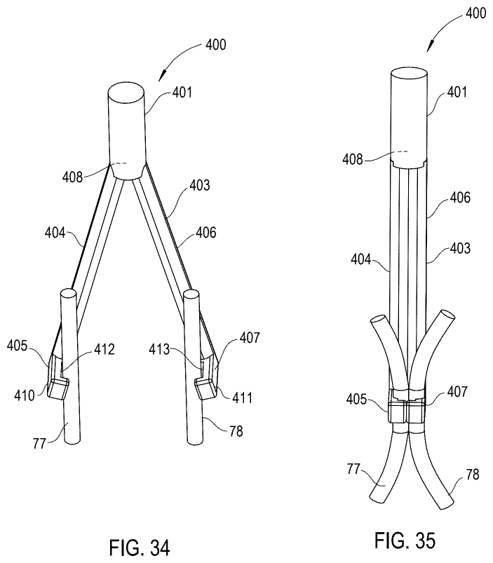

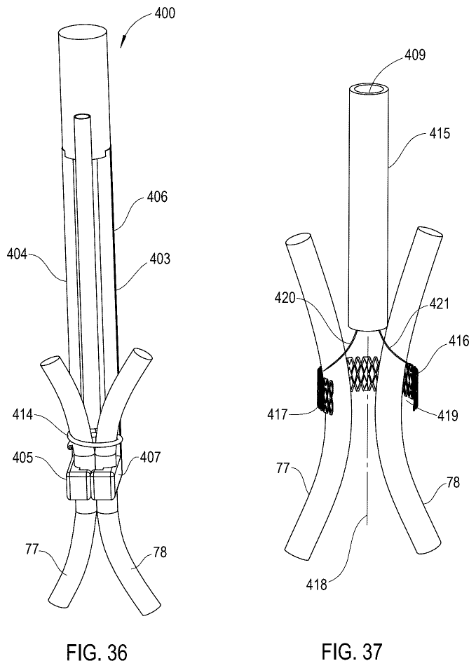

Turning to FIGS. 34-36, a system for grasping 400 comprising a catheter (or probe) 401 carries a forceps grasper 403 for engaging and approximating two or more chordae tendineae 77, 78. The grasper 403 includes a first elongate jaw 404 having a first engager 405 and a second elongate jaw 406 having a second engager 407. The jaws 404, 406 are pivotably articulatable in relation to each other via a pivot joint connection 408. The connection 408 can be moved distally and proximally within a lumen 409 (e.g., FIG. 37), or in alternative embodiments, the pivot joint can be operated by a micro gear motor within the catheter or probe. The engagers 405, 407 comprise transversely-extending arms 410, 411 each having an elongate undercut 412, 413. The catheter/probe 401 is delivered to the chordae tendineae 77, 78 and the desired chordae tendineae 77, 78 are placed between the open jaws 404, 406, as in FIG. 34. Then, with the undercuts 412, 413 grabbing the outside desired chordae tendineae 77, 78, the jaws 404, 406 are actuated by the actuator (manual or motorized/automated) to approximate the chordae tendineae 77, 78, as shown in FIG. 35. Once the desired chordae tendineae 77, 78 are approximated, a band 414 can be placed around the chordae tendineae 77, 78 to lock them in the approximated condition. Alternatively, as shown in FIG. 37, a catheter 415 having a lumen 409 is utilized to deliver a partial stent 416 around the approximated chordae tendineae 77, 78. The stent 416 comprises a partial perimeter 417, extending less than 360° around a longitudinal axis 418. In some embodiments, the partial perimeter 417 extends between about 200° and about 320° around the longitudinal axis 418. In some embodiments, the partial perimeter 417 extends between about 250° and about 300° around the longitudinal axis 418. The stent 416, because of its less than 360° perimeter 417, has a side opening 419 that allows it to be placed over the approximated chordae tendineae 77, 78 from the side. The stent 416 is then actuated (e.g., by adding heat, if the stent is an expanded shape-memory alloy stent with memory at a lesser diameter). The stent can then be detached from connecting wires 420, 421 by one of a number of detachment systems, including but not limited to electrolytic detachment mechanisms, pressurized detachment, hydraulic detachment mechanisms, mechanical or interlocking detachment mechanisms, heat-activated detachment systems, chemical detachment mechanisms, or frictional detachment systems. The stent 416, thus, locks the approximated chordae tendineae 77, 78 together. The stent 416 can comprise a mesh material, comprising a braid, or can comprise a laser-machined diamond cellular structure. In other embodiments, instead of being self-expanding, the stent 416 can be forcibly expanded via a balloon or other mechanical expansion structure.

In the embodiments of systems described in the present disclosure, treatment of the tricuspid valve can be performed, instead of the mitral valve. For example, when treating a tricuspid valve, one more catheters are inserted via a percutaneous puncture of a peripheral vein, or via a cut-down and puncture of the peripheral vein (e.g., femoral, subclavian, jugular) and delivery of the catheter(s) through the inferior vena cava and/or the superior vena cava and into the right atrium, and then through the tricuspid valve at least partially into the right ventricle. In other embodiments, instead of utilizing a percutaneous catheter approach, one or more probes can be placed through the heart wall, for example at the apex of the heart, and into the right ventricle.

Any of the catheters of the systems presented herein can comprise polymeric materials including, but not limited by, polyamide, polyethylene, polyurethane, or polyether block amide. In any of the previous embodiments that use wire comprising shape-memory alloy, alternative embodiments can be constructed using braided members. For example, braided disc-shaped members can be constructed. In some embodiments, the rings, bands, or hoops can comprise fabric material that comprises expanded fluoropolymer (ePTFE) fabric or polyester fabric. Any of the snares, bands, or other devices can comprise radiopaque materials for visualization on fluoroscopy or radiography. Any of the catheters can include a centering mechanism (inflatable balloon or expandable stent) that aids in crossing structures, such as the aortic valve, especially as the delivery catheter is caused to curve by the aortic arch. This also provides a stable leverage point for flexing the tip of the catheter when in the left ventricle.

Three major operations have been stated with the systems and methods presented. They are: the (1) grasping/snaring/engaging of the desired chordae tendineae; (2) the approximation of these grasped/snared/engaged chordae tendineae; and (3) the locking of the approximation. One or more of the embodiments described comprise an element and/or operation that performs (1) and (2). One or more of the embodiments described comprise an element and/or operation that performs (1), (2), and (3). When operations are performed together, they can all be performed from the same approach (e.g., arterial or venous). They can also be performed through the same sheath and/or guiding catheter (e.g., delivery catheter). When multiple operations involve two or more catheters or probes, they can each be delivered down the same delivery catheter (sheath or guiding catheter). Or, in certain cases, one or more of the catheters or probes can be delivered through a first delivery catheter and one or more other catheters or probes can be delivered down a second delivery catheter. And in some cases, or more other catheters or probes can be delivered through a third delivery catheter. Furthermore, two or more different delivery approaches can be utilized (e.g., arterial, and venous). Sheath or guiding catheter can comprise an outer diameter of 10 mm or less, or 9 mm or less, or 8 mm or less, or 7 mm or less, or between about 5 mm and about 10 mm, or between about 6 mm and about 9 mm.

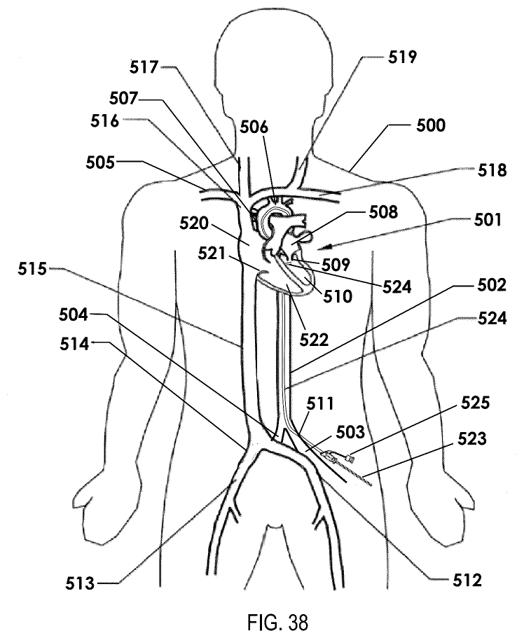

FIGS. 38-43 include the following labeled anatomical portions of a patient 500. Heart 501, descending aorta/aorta 502, left femoral artery 503, right femoral artery 504, right subclavian vein 505, aortic arch 506, ascending aorta 507, left atrium 508, mitral valve 509, left ventricle 510, arterial puncture site 511, left femoral vein 512, right femoral vein 513, venous puncture site 514, inferior vena cava 515, superior vena cava 516, right jugular vein 517, left subclavian vein 518, left jugular vein 519, right atrium 520, tricuspid valve 521, right ventricle 522.

FIG. 38 illustrates a first approach for performing procedures utilizing the systems and methods presented herein. The procedure is intended to treat a diseased mitral valve 509. A femoral artery (left femoral artery 503) is punctured with a hypodermic needle (not shown), using the Seldinger technique at the arterial puncture site 511, and a guidewire 523 is placed into the arterial system. The needle is removed, and a sheath is inserted into the artery. One or more arterial procedure catheter(s) 524 are placed through the aorta 502, aortic arch 506, ascending aorta 507, through the aortic valve, and at least partially into the left ventricle 510. All of the one or more arterial procedure catheter(s) 524 can be placed through one or more guiding catheters, and/or one or more sheaths. The one or more arterial procedure catheter(s) 524 include proximal connectors and/or controls 525 for manipulating and controlling them. Other arteries, such as a radial artery or an axillary artery can alternatively be chosen as a puncture or cut-down site.

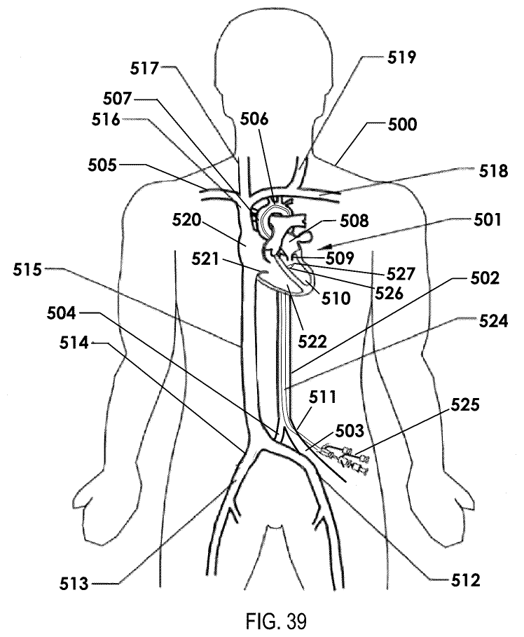

FIG. 39 illustrates a second approach for performing procedures utilizing the systems and methods presented herein. The procedure is intended to treat a diseased mitral valve 509. This approach is very similar to the one described in relation to FIG. 38, however the one or more arterial procedure catheter(s) 524 include at least one outer catheter 526 and at least one inner catheter 527 that is passed through a lumen of the outer catheter 526. In other specific approaches, this may be approved with a first catheter and a second catheter, utilized side-by-side. The approaches or FIGS. 38 and 39 are sometimes termed “transaortic delivery path.”

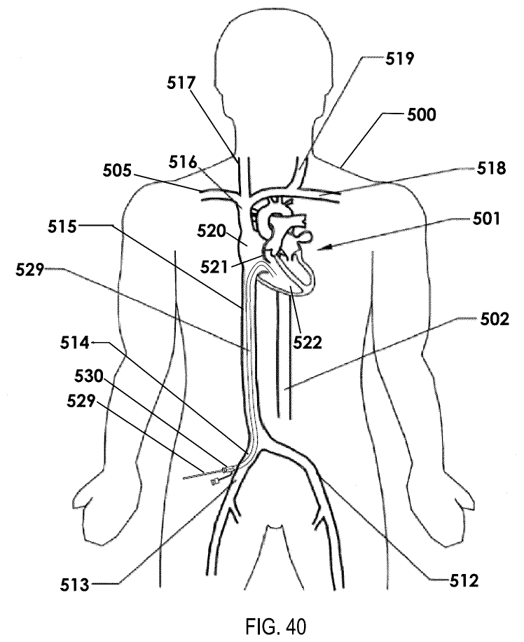

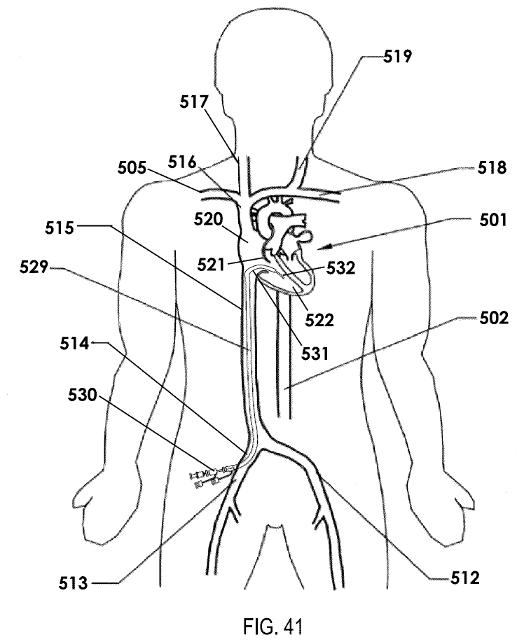

FIG. 40 illustrates a third approach for performing procedures utilizing the systems and methods presented herein. The procedure is intended to treat a diseased tricuspid valve 521. A femoral vein (right femoral vein 513) is punctured with a hypodermic needle (not shown), using the Seldinger technique at the venous puncture site 514, and a guidewire 529 is placed into the venous system. The needle is removed, and a sheath is inserted into the vein. One or more venous procedure catheter(s) 529 are placed through the inferior vena cava 515, right atrium 520, and past the diseased tricuspid valve 521 at least partially into the right ventricle 522. All of the one or more venous procedure catheter(s) 529 can be placed through one or more guiding catheters, and/or one or more sheaths. The one or more venous procedure catheter(s) 529 include proximal connectors and/or controls 530 for manipulating and controlling them.

FIG. 41 illustrates a fourth approach for performing procedures utilizing the systems and methods presented herein. The procedure is intended to treat a diseased tricuspid valve 521. This approach is very similar to the one described in relation to FIG. 40, however the one or more venous procedure catheter(s) 529 include at least one catheter 531 whose distal tip remains within the right atrium 520, and at least one catheter 532 that is passed through the diseased tricuspid valve 521, and into the right ventricle 522.

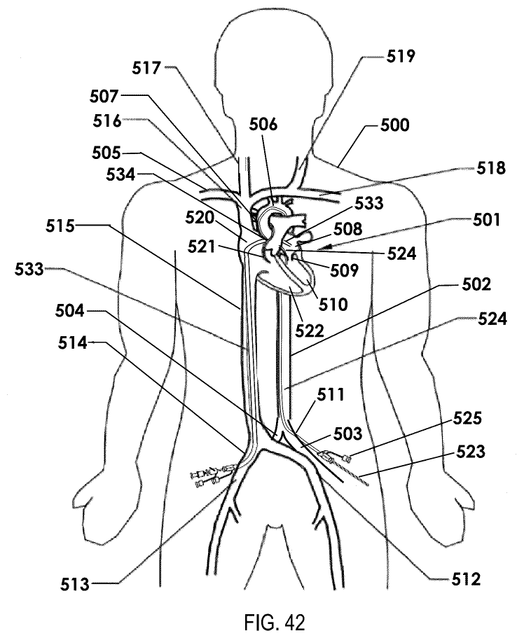

FIG. 42 illustrates a fifth approach for performing procedures utilizing the systems and methods presented herein. The procedure is intended to treat a diseased mitral valve 509, but uses a tandem arterial and venous approach. The arterial catheters are utilized in a similar manner as that described in relation with FIG. 38 or FIG. 39. However, a different set of venous catheters 533 are utilized, including catheters to puncture and cross the septum 534 between the right atrium 520 and the left atrium 508. The one or more arterial procedure catheter(s) 524 can be placed through one or more guiding catheters, and/or one or more sheaths. The one or more venous procedure catheter(s) 533 can be placed through one or more guiding catheters, and/or one or more sheaths. The venous-delivered catheters can be shaped with a 90° bend or have the ability to flex in this manner, in order to fit into the trans-mitral delivery after passing through the septal puncture. Furthermore, any of the arterial-delivered catheters can be shaped with a 90° bend or have the ability to flex in this manner. Returning to FIG. 41, a tandem procedure for treatment of the tricuspid valve 521 is also possible. In this procedure, one or more catheters are placed venously via the (first) venous puncture site 514, and advanced through the inferior vena cava 515 to the right heart. A second one or more catheters are placed venously via a second venous puncture site (e.g., left subclavian vein 518, left jugular vein 519, right subclavian vein 505, right jugular vein 517) and advanced through the superior vena cava 516 to the right heart.

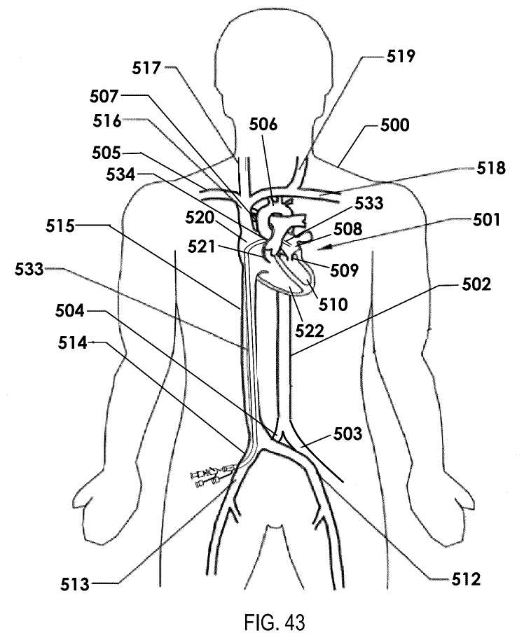

FIG. 43 illustrates a sixth approach for performing procedures utilizing the systems and methods presented herein. The procedure is intended to treat a diseased mitral valve 509, but uses a purely venous approach. The venous catheters 533 are utilized, including catheters to puncture and cross the septum 534 between the right atrium 520 and the left atrium 508. The one or more venous procedure catheter(s) 533 can be placed through one or more guiding catheters, and/or one or more sheaths. The venous-delivered catheters can be shaped with a 90° bend or have the ability to flex in this manner, in order to fit into the trans-mitral delivery after passing through the septal puncture.

Any of the catheters described herein can be used in any of the approaches and combinations described in relation to FIGS. 38-43.

As described, alternatively, catheters or probes can deliver any of the embodiments described above via a trans-myocardial approach, for example through the myocardia at the heart apex, and into the ventricle. This can be performed via a surgically opened chest with the sternum cut. Alternatively, the chest wall can be passed via a puncture (e.g., laparoscopic using trocars).

In some patients, one or more chord may have become severely stretched or ruptured. It is possible, thus, to use any of the systems and methods described herein to tether this chord in parallel to another chord, to bring back some benefit from the damaged chord.

Systolic anterior motion of the anterior mitral leaflet, in which the leaflet is shifted toward the left ventricular outflow tract during valve closure, can occur following surgical implantation of an annuloplasty ring. The chordal approximation of chords from both papillary muscles along with a connecting string or wire, as described herein, can be used connect the anterior chords to posterior strut chords and pull the anterior leaflet posteriorly, and thus prevent the outflow tract blockage.

Finally, although the systems and methods disclosed generally act on the chordae alone, it is possible to used any of these methods to act on the very lower part of the chordae, adjacent to the papillary muscle; or, alternatively, to include to action to the papillary muscle itself.

Certain embodiments of the present disclosure are described in the following clauses:

Clause 1—A method for treating a patient includes delivering a distal portion of an elongate medical device into a ventricle of a beating closed heart of a subject via a primary skin opening and a secondary puncture; utilizing the distal portion of the elongate medical device to engage a plurality of chordae tendineae extending from a first papillary muscle within the ventricle, at least a first one of the plurality of chordae tendineae attached to a first leaflet of an atrioventricular valve located between the ventricle and an adjacent atrium, and at least a second one of the plurality of chordae tendineae attached to a second leaflet of the atrioventricular valve; approximating the engaged plurality of chordae tendineae to increase an amount of closure possible between the first leaflet and the second leaflet; and locking the plurality of chordae tendineae in their approximated condition.

Clause 2—The method of clause 1, wherein the atrioventricular valve includes a mitral valve and the ventricle includes a left ventricle.

Clause 3—The method of clause 1, wherein the atrioventricular valve includes a tricuspid valve and the ventricle includes a right ventricle.

Clause 4—The method of clause 3, wherein the first leaflet includes an anterior leaflet, and wherein the second leaflet includes a posterior leaflet.

Clause 5—The method of clause 3, wherein the first leaflet includes an anterior leaflet, and wherein the second leaflet includes a septal leaflet.

Clause 6—The method of clause 3, wherein the first leaflet includes an anterior leaflet, and wherein the second leaflet includes a septal leaflet.

Clause 7—The method of any one of clauses 1-6, further including engaging a second plurality of chordae tendineae extending from a second papillary muscle within the ventricle, at least a first one of the second plurality of chordae tendineae attached to the first leaflet of the atrioventricular valve, and at least a second one of the second plurality of chordae tendineae attached to the second leaflet of the atrioventricular valve; approximating the engaged second plurality of chordae tendineae to further increase the amount of closure possible between the first leaflet and the second leaflet; and locking the second plurality of chordae tendineae in their approximated condition.

Clause 8—The method of clause 1, wherein the ventricle includes a left ventricle, and the secondary puncture includes a puncture of an artery.

Clause 9—The method of clause 8, wherein the artery includes a femoral artery.

Clause 10—The method of clause 1, wherein the ventricle includes a left ventricle, and wherein the secondary puncture includes a puncture of a vein.

Clause 11—The method of clause 10, wherein the vein includes a femoral vein.

Clause 12—The method of clause 10, wherein delivering a distal portion of an elongate medical device into a ventricle includes delivering the distal portion through a tertiary puncture in a septal wall between a right artium and a left atrium.

Clause 13—The method of clause 1, wherein the ventricle includes a right ventricle, and wherein the secondary puncture includes a puncture of a vein.

Clause 14—The method of clause 13, wherein the vein includes a femoral vein.

Clause 15—The method of clause 13, wherein the vein includes a jugular vein.

Clause 16—The method of clause 13, wherein the vein includes a subclavian vein.

Clause 17—The method of clause 1, wherein the ventricle includes a left ventricle, and wherein the secondary puncture includes a puncture in an apex of the heart.

Clause 18—The method of clause 1, wherein the ventricle includes a right ventricle, and wherein the secondary puncture includes a puncture in an apex of the heart.

Clause 19—The method of any one of clauses 1-6 and 8-18, wherein the primary skin opening includes a puncture in the skin.

Clause 20—The method of clause 7, wherein the primary skin opening includes an incision in the skin.

Clause 21—The method of any one of clauses 1-6 and 8-18, wherein the primary skin opening includes an incision in the skin.

Clause 22—The method of clause 1, wherein the first papillary muscle includes an anterolateral papillary muscle.

Clause 23—The method of clause 1, wherein the first papillary muscle includes an posteromedial papillary muscle.

Clause 24—The method of any one of clauses 1-23, further including assessing the amount of coaptation between the first leaflet and the second leaflet.

Clause 25—The method of clauses 24, wherein assessing is performed with echocardiography.

Clause 26—The method of clauses 25, wherein assessing is performed with transthoracic echocardiography.

Clause 27—The method of any one of clauses 1-6, wherein the approximating proceeds substantially along an anterior-posterior axis.

Clause 28—The method of any one of clauses 1-6, wherein engaging includes snaring with a snare.

Clause 29—The method of any one of clauses 1-6, wherein engaging includes encircling.

Clause 30—The method of any one of clauses 1-6, wherein engaging includes grasping with a grasper.

Clause 31—The method of any one of clauses 1-6, wherein approximating includes reducing a distance between the at least a first one of the plurality of chordae tendineae and the at least a second one of the plurality of chordae tendineae.

Clause 32—The method of clause 31, wherein approximating includes reducing a distance between the at least a first one of the plurality of chordae tendineae and the at least a second one of the plurality of chordae tendineae.

Clause 33—The method of clause 32, wherein locking includes placing a constricting band around the at least a first one of the plurality of chordae tendineae and the at least a second one of the plurality of chordae tendineae.

Clause 34—The method of any one of clauses 1-6, wherein locking includes placing a constrictor around the at least a first one of the plurality of chordae tendineae and the at least a second one of the plurality of chordae tendineae.

Clause 35—The method of any one of clauses 1-6, wherein locking includes placing a metal loop around the at least a first one of the plurality of chordae tendineae and the at least a second one of the plurality of chordae tendineae.

Clause 36—The method of any one of clauses 1-6, wherein locking includes placing a clip around the at least a first one of the plurality of chordae tendineae and the at least a second one of the plurality of chordae tendineae.

Clause 37—The method of any one of clauses 1-6, wherein locking includes placing a stent around the at least a first one of the plurality of chordae tendineae and the at least a second one of the plurality of chordae tendineae.

Clause 38—The method of clause 37, wherein the stent includes a cylinder having a continuous 360° perimeter around a longitudinal axis.

Clause 39—The method of clause 37, wherein the stent includes a partial perimeter, extending less than 360° around a longitudinal axis.

Clause 40—The method of clause 39, wherein the partial perimeter extends between about 200° and about 320° around the longitudinal axis, or between about 250° and about 300° around the longitudinal axis.

Clause 41—A method for treating a patient includes non-surgically engaging a plurality of chordae tendineae extending from a first papillary muscle within a ventricle, at least a first one of the plurality of chordae tendineae attached to a first leaflet of an atrioventricular valve located between the ventricle and an adjacent atrium, and at least a second one of the plurality of chordae tendineae attached to a second leaflet of the atrioventricular valve; non-surgically approximating the engaged plurality of chordae tendineae to increase an amount of closure possible between the first leaflet and the second leaflet; and non-surgically locking the plurality of chordae tendineae in their approximated condition.

Clause 42—The method of clause 41, wherein non-surgically engaging is performed with a first elongate medical device.

Clause 43—The method of clause 42, wherein non-surgically approximating is performed with the first elongate medical device.

Clause 44—The method of clause 42, wherein non-surgically approximating is performed with a second elongate medical device.

Clause 45—The method of clause 44, wherein non-surgically locking is performed with a third elongate medical device.

Clause 46—The method of clause 44, wherein non-surgically locking is performed with the second elongate medical device.

Clause 47—The method of clause 44, wherein non-surgically locking is performed with the first elongate medical device.

Clause 48—The method of clause 44, wherein the ventricle includes a left ventricle, and wherein the first elongate medical device and the second elongate medical device are both inserted through a delivery lumen of an elongate delivery device.

Clause 49—The method of clause 48, wherein the elongate delivery device includes a catheter.

Clause 50—The method of either one of clauses 48 or 49, wherein the elongate delivery device is inserted through an artery and delivered to the ascending aorta of the subject.

Clause 51—The method of either one of clauses 48 or 49, wherein the elongate delivery device is inserted through a vein and delivered to the left ventricle of the subject through a puncture in a septal wall between a right artium and a left atrium.

Clause 52—The method of clause 45, wherein the ventricle includes a left ventricle, and wherein the first elongate medical device, the second elongate medical device, and the third elongate medical device are all inserted through a delivery lumen of an elongate delivery device.

Clause 53—The method of clause 52, wherein the elongate delivery device includes a catheter.

Clause 54—The method of either one of clauses 52 or 53, wherein the elongate delivery device is inserted through an artery and delivered to the ascending aorta of the subject.

Clause 55—The method of either one of clauses 52 or 53, wherein the elongate delivery device is inserted through a vein and delivered to the left ventricle of the subject through a puncture in a septal wall between a right artium and a left atrium.

Clause 56—The method of clause 44, wherein the ventricle includes a right ventricle, and wherein the first elongate medical device and the second elongate medical device are both inserted through a delivery lumen of an elongate delivery device.

Clause 57—The method of clause 56, wherein the elongate delivery device includes a catheter.

Clause 58—The method of either one of clauses 56 or 57, wherein the elongate delivery device is inserted through a vein.

Clause 59—The method of clause 45, wherein the ventricle includes a right ventricle, and wherein the first elongate medical device, the second elongate medical device, and the third elongate medical device are all inserted through a delivery lumen of an elongate delivery device.

Clause 60—The method of clause 59, wherein the elongate delivery device includes a catheter.

Clause 61—The method of either one of clauses 59 or 60, wherein the elongate delivery device is inserted through a vein.

Clause 62—The method of clause 44, wherein the ventricle includes a left ventricle, wherein the first elongate medical device is inserted through a delivery lumen of a first elongate delivery device and wherein the second elongate medical device is inserted through a delivery lumen of a second elongate delivery device.

Clause 63—The method of clause 62, wherein the first elongate delivery device includes a first catheter and wherein the second elongate delivery device includes a second catheter.

Clause 64—The method of either one off clauses 62 or 63, wherein the first elongate delivery device is inserted through an artery and delivered to the ascending aorta of the subject, and wherein the second elongate delivery device is inserted through a vein and delivered to the left ventricle of the subject through a puncture in a septal wall between a right artium and a left atrium.

Clause 65—The method of either one of clauses 62 or 63, wherein the second elongate delivery device is inserted through an artery and delivered to the ascending aorta of the subject, and wherein the first elongate delivery device is inserted through a vein and delivered to the left ventricle of the subject through a puncture in a septal wall between a right artium and a left atrium.

Clause 66—The method of either one of clauses 43 or 44, wherein non-surgically locking is performed with an elongate locking device.

Clause 67—The method of clause 66, wherein the ventricle includes a left ventricle, wherein the first elongate medical device is inserted through a delivery lumen of a first elongate delivery device and wherein the elongate locking device is inserted through a delivery lumen of a second elongate delivery device.

Clause 68—The method of clause 67, wherein the first elongate delivery device includes a first catheter and wherein the second elongate delivery device includes a second catheter.

Clause 69—The method of either one of clauses 67 or 68, wherein the first elongate delivery device is inserted through an artery and delivered to the ascending aorta of the subject, and wherein the second elongate delivery device is inserted through a vein and delivered to the left ventricle of the subject through a puncture in a septal wall between a right artium and a left atrium.

Clause 70—The method of clause 69, wherein one or both of the first elongate medical device and/or the second elongate medical device is/are delivered across the a mitral valve of the subject.

Clause 71—A system includes a catheter including an elongate shaft having a distal end and a proximal end and configured to be advanced into a ventricle of a heart of a subject; a grasper carried by the distal end of the shaft and configured to engage at least a first one of a plurality of chordae tendineae extending from a papillary muscle within the ventricle and attached to a first leaflet of an atrioventricular valve located between the ventricle and an adjacent atrium, and to engage at least a second one of the plurality of chordae tendineae attached to a second leaflet of the atrioventricular valve, and to further maintain simultaneous engagement of both of the at least a first one of the plurality of chordae tendineae and the at least a second one of the plurality of chordae tendineae; an actuator carried by the shaft and configured to cause the at least a first one of the plurality of chordae tendineae and the at least a second one of the plurality of chordae tendineae to be moved toward each other; and a fastener carried by the distal end of the shaft and configured to lock the at least a first one of the plurality of chordae tendineae and the at least a second one of the plurality of chordae tendineae with respect to each other.

Clause 72—The system of clause 71, wherein the grasper includes an elongate line including a first longitudinally-extending line portion having a distal end and a proximal end, a second longitudinally-extending line portion having a distal end and a proximal end, and a turn connecting the distal end of the first longitudinally-extending line portion to the second longitudinally-extending line portion, wherein the first longitudinally-extending line portion and the second longitudinally-extending line portion are configured to be held substantially parallel to each other.

Clause 73—The system of clause 72, wherein the turn includes a 180° turn.

Clause 74—The system of clause 72, wherein the turn includes a partial circular shape.

Clause 75—The system of clause 74, wherein the first longitudinally-extending line portion and the second longitudinally-extending line portion are separated by a distance d, and wherein the circular shape has an inner diameter D that is greater than the distance d.

Clause 76—The system of clause 75, wherein the partial circular shape extends over a curve of between 200° and 340°.

Clause 77—The system of clause 76, wherein the elongate line further includes a first laterally-facing concave curve between the partial circular shape and the distal end of the first longitudinally-extending line portion and a second laterally-facing concave curve between the partial circular shape and the distal end of the second longitudinally-extending line portion.

Clause 78—The system of any one of clauses 72-77, wherein the line includes a shape-memory alloy.

Clause 79—The system of any one of clauses 72-77, wherein the line includes a monofilament strand.

Clause 80—The system of any one of clauses 72-77, wherein the line includes a braided strand.

Clause 81—The system of any one of clauses 72-77, wherein the line includes a hollow tube.

Clause 82—The system of clause 81, wherein the hollow tube includes a shape-memory alloy.

Clause 83—The system of clause 81, wherein the hollow tube includes a polymer.

Clause 84—The system of any one of clauses 72-77, wherein the line includes a metallic material having a polymeric covering.

Clause 85—The system of clause 84, wherein the covering includes material selected from the list consisting of: PTFE, ETFE, FEP, PFE, polyethylene, and polypropylene.

Clause 86—The system of clause 84, wherein the covering includes material selected from the list consisting of: PTFE, ETFE, FEP, PFE, polyethylene, and polypropylene.

Clause 87—The system of clause 75, wherein the actuator includes a hollow slide having an inner opening passing therethrough, the inner opening having an inner diameter configured to allow the passage of the first longitudinally-extending line portion and the second longitudinally-extending line portion simultaneously.

Clause 88—The system of clause 87, wherein the hollow slide includes a tube having a lumen, wherein the inner diameter is the diameter of the lumen.

Clause 89—The system of clause 87, wherein the tube includes a shaft of the catheter.

Clause 90—The system of clause 87, wherein the tube is slideable within an elongate catheter lumen of the catheter.

Clause 91—The system of clause 90, wherein the catheter further includes an elongate mandrel coupled to the tube and configured to push the tube distally within the catheter lumen.

Clause 92—The system of clause 91, wherein the elongate mandrel is further configured to pull the tube proximally within the catheter lumen.

Clause 93—The system of clause 90, wherein the hollow slide is configured to be slid distally such that the lumen is forced over a proximal portion of the partial circular shape, causing the inner diameter D to be decreased.

Clause 94—The system of clause 87, wherein the hollow slide is configured to be slid distally such that the lumen is forced over a proximal portion of the partial circular shape, causing the inner diameter D to be decreased.

Clause 95—The system of clause 75, wherein the actuator includes a slide located at a proximal end of the catheter and configured to retract one or both of the first longitudinally-extending line portion and/or the second longitudinally-extending line portion of the elongate line, to cause the inner diameter D to be decreased.

Clause 96—The system of clause 95, wherein the catheter includes a catheter lumen, and wherein pulling the partial circular shape at least partially into the catheter lumen causes the decrease in the inner diameter D of the circular shape.

Clause 97—The system of clause 71, further including an elongate line including a first longitudinally-extending line portion having a distal end and a proximal end, a second longitudinally-extending line portion having a distal end and a proximal end, and a turn connecting the distal end of the first longitudinally-extending line portion to the second longitudinally-extending line portion, wherein the first longitudinally-extending line portion and the second longitudinally-extending line portion are configured to be held substantially parallel to each other, wherein the grasper includes the turn, and wherein the actuator includes at least a proximal portion of the first longitudinally-extending line portion and a proximal portion of the second longitudinally-extending line portion, wherein user-applied traction on the a proximal portion of the first longitudinally-extending line portion and the proximal portion of the second longitudinally-extending line portion is configured to aid the closing of the turn.

Clause 98—The system of clause 97, wherein the actuator further includes a cover configured to be slid over the turn, to cause the turn to close over the at least a first one of the plurality of chordae tendineae and the at least a second one of the plurality of chordae tendineae.

Clause 99—The system of clause 98, wherein the cover includes a constraining band configured to be slid over the elongate line.

Clause 100—The system of clause 99, wherein the cover includes a shape-memory alloy, and has a ring shape.

Clause 101—The system of clause 100, wherein the cover is capable of forming a stress-induced Martensite shape that is substantially straight as the cover is slid over one or both of the first longitudinally-extending line portion and/or the proximal portion of the second longitudinally-extending line portion.

Clause 102—The system of either one of clauses 100 or 101, wherein the cover moves toward the ring shape as the cover moves toward an Austenitic shape.

Clause 103—The system of clause 102, wherein the cover includes an inner channel configured to slidingly engage the line.

Clause 104—The system of clause 102, wherein the cover includes an inner lumen configured to slidingly engage the line.

Clause 105—The system of clause 102, wherein the cover in its stress-induced Martensite shape includes the actuator, and wherein the cover at or near its Austenitic shape includes the fastener.

Clause 106—The system of any one of clauses 72-77 or 97-101, wherein one or both of the first longitudinally-extending line portion and/or the second longitudinally-extending line portion is/are configured to be detached from the turn.

Clause 107—The system of any one of clauses 71-77 or 97, wherein the fastener includes a band.

Clause 108—The system of clause 107, wherein the band includes a ring of flat shape-memory alloy.

Clause 109—The system of clause 107, wherein the band includes a spiral.

Clause 110—The system of clause 107, wherein the band includes a coil.

Clause 111—The system of clause 107, wherein the band does not include a continuous 360° ring.

Clause 112—The system of clause 111, wherein the band extends over a curve of between 200° and 340°.

Clause 113—The system of clause 112, wherein the band extends over a curve of between 240° and 300°.

Clause 114—The system of clause 111, wherein the band includes a first end and a second end, the first end and the second end inverted within an interior of the band.

Clause 115—The system of clause 111, wherein the band includes a first end and a second end, the first end and the second end everted outside an exterior of the band.

Clause 116—The system of clause 107, wherein the band includes a coil that extends over a curve of between 365° and 720°.

Clause 117—The system of clause 107, wherein the band includes a laser-machined portion.

Clause 118—The system of clause 117, wherein the laser-machined portion includes a serpentine shape.

Clause 119—The system of clause 118, wherein the serpentine shape is a squared serpentine shape.

Clause 120—The system of any one of clauses 107-119, wherein the band has a longitudinal axis that is configured to be oriented substantially perpendicular to the shaft of the catheter.

Clause 121—The system of any one of clauses 107-119, wherein the band has a longitudinal axis that is configured to be oriented substantially parallel to the shaft of the catheter.

Clause 122—The system of clause 107, wherein the band is formed from two curved arms.

Clause 123—The system of clause 122, wherein the two curved arms have at least some radial overlap.

Clause 124—The system of clause 122, wherein the two curved arms include elongate flat metallic arms that are curved.

Clause 125—The system of clause 107, wherein the band is formed from a wire.

Clause 126—The system of clause 125, wherein the wire includes a discontinuity. Clause 127—The system of clause 107, wherein the band includes a stent.

Clause 128—The system of clause 127, wherein the stent is braided.

Clause 129—The system of clause 127, wherein the stent is laser-machined.

Clause 130—The system of clause 129, wherein the stent has a zig-zag pattern.

Clause 131—The system of clause 71, wherein the grasper includes a pair of opposing hooks.

Clause 132—The system of clause 71, wherein the grasper includes a pair of opposing jaws.

Clause 133—The system of either one of clauses 131 or 132, wherein the pair is arrayed longitudinally.

Clause 134—The system of clause 133, wherein the pair includes a proximal element and a distal element slidable in relation to each other.

Clause 135—The system of clause 134, wherein one of the proximal and distal element is carried on a tube and the other of the proximal element and distal element is carried on an elongate shaft that is slidable within the tube.

Clause 136—The system of clause 133, wherein the pair are configured to engage each other.

Clause 137—The system of clause 136, wherein the pair are configured to snap together.

Clause 138—The system of either one of clauses 131 or 132, wherein the pair is arrayed laterally.

Clause 139—The system of clause 138, wherein the pair includes a left element and a right element.

Clause 140—The system of clause 139, wherein the pair includes a pivot joint between the left element and the right element that allows the left element and the right element to close toward each other.

Clause 141—The system of clause 140, further including a longitudinal passageway extending between the left element and the right element.

Clause 142—The system of any one of clauses 131-141, wherein the grasper in a modified orientation, state, or position includes the fastener.

Clause 143—The system of clause 71, wherein the fastener includes a pair of opposing hooks.

Clause 144—The system of clause 71, wherein the fastener includes a pair of opposing jaws.

Clause 145—The system of either one of clauses 143 or 144, wherein the pair is arrayed longitudinally.

Clause 146—The system of clause 145, wherein the pair includes a proximal element and a distal element slidable in relation to each other.

Clause 147—The system of clause 146, wherein one of the proximal and distal element is carried on a tube and the other of the proximal element and distal element is carried on an elongate shaft that is slidable within the tube.

Clause 148—The system of clause 145, wherein the pair are configured to engage each other.

Clause 149—The system of clause 148, wherein the pair are configured to snap together.

Clause 150—The system of either one of clauses 143 or 144, wherein the pair is arrayed laterally.

Clause 151—The system of clause 150, wherein the pair includes a left element and a right element.

Clause 152—The system of clause 151, wherein the pair includes a pivot joint between the left element and the right element that allows the left element and the right element to close toward each other.

Clause 153—The system of clause 152, further including a longitudinal passageway extending between the left element and the right element.

Clause 154—A system includes a catheter including an elongate shaft having a distal end and a proximal end and configured to be advanced into a ventricle of a heart of a subject; a grasper carried by the distal end of the shaft and configured to engage at least a first one of a plurality of chordae tendineae extending from a papillary muscle within the ventricle and attached to a first leaflet of an atrioventricular valve located between the ventricle and an adjacent atrium, and to engage at least a second one of the plurality of chordae tendineae attached to a second leaflet of the atrioventricular valve, and to further maintain simultaneous engagement of both of the at least a first one of the plurality of chordae tendineae and the at least a second one of the plurality of chordae tendineae; an actuator carried by the shaft and configured to cause the at least a first one of the plurality of chordae tendineae and the at least a second one of the plurality of chordae tendineae to be moved toward each other; and an energy source carried by the distal end of the shaft and configured to deliver energy to fix the at least a first one of the plurality of chordae tendineae and the at least a second one of the plurality of chordae tendineae in place relative to each other.

Clause 155—The system of clause 154, wherein the grasper includes an elongate line including a first longitudinally-extending line portion having a distal end and a proximal end, a second longitudinally-extending line portion having a distal end and a proximal end, and a turn connecting the distal end of the first longitudinally-extending line portion to the second longitudinally-extending line portion, wherein the first longitudinally-extending line portion and the second longitudinally-extending line portion are configured to be held substantially parallel to each other.

Clause 156—The system of clause 155, wherein the turn includes a 180° turn.

Clause 157—The system of clause 155, wherein the turn includes a partial circular shape.

Clause 158—The system of clause 157, wherein the first longitudinally-extending line portion and the second longitudinally-extending line portion are separated by a distance d, and wherein the circular shape has an inner diameter D that is greater than the distance d.

Clause 159—The system of clause 158, wherein the partial circular shape extends over a curve of between 200° and 340°.

Clause 160—The system of clause 159, wherein the elongate line further includes a first laterally-facing concave curve between the partial circular shape and the distal end of the first longitudinally-extending line portion and a second laterally-facing concave curve between the partial circular shape and the distal end of the second longitudinally-extending line portion.

Clause 161—The system of any one of clauses 155-160, wherein the line includes a shape-memory alloy.

Clause 162—The system of any one of clauses 155-160, wherein the line includes a monofilament strand.

Clause 163—The system of any one of clauses 155-160, wherein the line includes a braided strand.

Clause 164—The system of any one of clauses 155-160, wherein the line includes a hollow tube.

Clause 165—The system of clause 164, wherein the hollow tube includes a shape-memory alloy.

Clause 166—The system of clause 164, wherein the hollow tube includes a polymer.

Clause 167—The system of any one of clauses 155-160, wherein the line includes a metallic material having a polymeric covering.

Clause 168—The system of clause 167, wherein the covering includes a coating.

Clause 169—The system of clause 167, wherein the covering includes material selected from the list consisting of: PTFE, ETFE, FEP, PFE, polyethylene, and polypropylene.

Clause 170—The system of clause 158, wherein the actuator includes a hollow slide having an inner opening passing therethrough, the inner opening having an inner diameter configured to allow the passage of the first longitudinally-extending line portion and the second longitudinally-extending line portion simultaneously.

Clause 171—The system of clause 170, wherein the hollow slide includes a tube having a lumen, wherein the inner diameter is the diameter of the lumen.

Clause 172—The system of clause 170, wherein the tube includes a shaft of the catheter.

Clause 173—The system of clause 170, wherein the tube is slideable within an elongate catheter lumen of the catheter.

Clause 174—The system of clause 173, wherein the catheter further includes an elongate mandrel coupled to the tube and configured to push the tube distally within the catheter lumen.

Clause 175—The system of clause 174, wherein the elongate mandrel is further configured to pull the tube proximally within the catheter lumen.

Clause 176—The system of clause 173, wherein the hollow slide is configured to be slid distally such that the lumen is forced over a proximal portion of the partial circular shape, causing the inner diameter D to be decreased.

Clause 177—The system of clause 170, wherein the hollow slide is configured to be slid distally such that the lumen is forced over a proximal portion of the partial circular shape, causing the inner diameter D to be decreased.

Clause 178—The system of clause 158, wherein the actuator includes a slide located at a proximal end of the catheter and configured to retract one or both of the first longitudinally-extending line portion and/or the second longitudinally-extending line portion of the elongate line, to cause the inner diameter D to be decreased.

Clause 179—The system of clause 178, wherein the catheter includes a catheter lumen, and wherein pulling the partial circular shape at least partially into the catheter lumen causes the decrease in the inner diameter D of the circular shape.

Clause 180—The system of clause 154, further including an elongate line including a first longitudinally-extending line portion having a distal end and a proximal end, a second longitudinally-extending line portion having a distal end and a proximal end, and a turn connecting the distal end of the first longitudinally-extending line portion to the second longitudinally-extending line portion, wherein the first longitudinally-extending line portion and the second longitudinally-extending line portion are configured to be held substantially parallel to each other, wherein the grasper includes the turn, and wherein the actuator includes at least a proximal portion of the first longitudinally-extending line portion and a proximal portion of the second longitudinally-extending line portion, wherein user-applied traction on the a proximal portion of the first longitudinally-extending line portion and the proximal portion of the second longitudinally-extending line portion is configured to aid the closing of the turn.

Clause 181—The system of clause 180, wherein the actuator further includes a cover configured to be slid over the turn, to cause the turn to close over the at least a first one of the plurality of chordae tendineae and the at least a second one of the plurality of chordae tendineae.

Clause 182—The system of clause 181, wherein the cover includes a constraining band configured to be slid over the elongate line.

Clause 183—The system of clause 182, wherein the cover includes a shape-memory alloy, and has a ring shape.

Clause 184—The system of clause 183, wherein the cover is capable of forming a stress-induced Martensite shape that is substantially straight as the cover is slid over one or both of the first longitudinally-extending line portion and/or the proximal portion of the second longitudinally-extending line portion.

Clause 185—The system of either one of clauses 183 or 184, wherein the cover moves toward the ring shape as the cover moves toward an Austenitic shape.

Clause 186—The system of clause 185, wherein the cover includes an inner channel configured to slidingly engage the line.

Clause 187—The system of clause 185, wherein the cover includes an inner lumen configured to slidingly engage the line.

Clause 188—The system of any one of clauses 155-160 or 180-184, wherein one or both of the first longitudinally-extending line portion and/or the second longitudinally-extending line portion is/are configured to be detached from the turn.

Clause 189—The system of clause 182, wherein the band includes a ring of flat shape-memory alloy.

Clause 190—The system of clause 182, wherein the band includes a spiral.

Clause 191—The system of clause 182, wherein the band includes a coil.

Clause 192—The system of clause 182, wherein the band does not include a continuous 360° ring.

Clause 193—The system of clause 192, wherein the band extends over a curve of between 200° and 340°.

Clause 194—The system of clause 193, wherein the band extends over a curve of between 240° and 300°.

Clause 195—The system of clause 192, wherein the band includes a first end and a second end, the first end and the second end inverted within an interior of the band.

Clause 196—The system of clause 192, wherein the band includes a first end and a second end, the first end and the second end everted outside an exterior of the band.

Clause 197—The system of clause 182, wherein the band includes a coil that extends over a curve of between 365° and 720°.

Clause 198—The system of clause 182, wherein the band includes a laser-machined portion.

Clause 199—The system of clause 198, wherein the laser-machined portion includes a serpentine shape.

Clause 200—The system of clause 199, wherein the serpentine shape is a squared serpentine shape.

Clause 201—The system of any one of clauses 182-200, wherein the band has a longitudinal axis that is configured to be oriented substantially perpendicular to the shaft of the catheter.

Clause 202—The system of any one of clauses 182-200, wherein the band has a longitudinal axis that is configured to be oriented substantially parallel to the shaft of the catheter.

Clause 203—The system of clause 182, wherein the band is formed from two curved arms.

Clause 204—The system of clause 203, wherein the two curved arms have at least some radial overlap.

Clause 205—The system of clause 203, wherein the two curved arms include elongate flat metallic arms that are curved.

Clause 206—The system of clause 182 wherein the band is formed from a wire.

Clause 207—The system of clause 206, wherein the wire includes a discontinuity. Clause 208—The system of clause 182, wherein the band includes a stent.

Clause 209—The system of clause 208, wherein the stent is braided.

Clause 210—The system of clause 208, wherein the stent is laser-machined.

Clause 211—The system of clause 210, wherein the stent has a zig-zag pattern.

Clause 212—The system of clause 154, wherein the grasper includes a pair of opposing hooks.

Clause 213—The system of clause 154, wherein the grasper includes a pair of opposing jaws.

Clause 214—The system of either one of clauses 212 or 213, wherein the pair is arrayed longitudinally.

Clause 215—The system of clause 214, wherein the pair includes a proximal element and a distal element slidable in relation to each other.

Clause 216—The system of clause 215, wherein one of the proximal and distal element is carried on a tube and the other of the proximal element and distal element is carried on an elongate shaft that is slidable within the tube.

Clause 217—The system of clause 214, wherein the pair are configured to engage each other.

Clause 218—The system of clause 217, wherein the pair are configured to snap together.

Clause 219—The system of either one of clauses 212 or 213, wherein the pair is arrayed laterally.

Clause 220—The system of clause 219, wherein the pair includes a left element and a right element.

Clause 221—The system of clause 220, wherein the pair includes a pivot joint between the left element and the right element that allows the left element and the right element to close toward each other.

Clause 222—The system of clause 221, further including a longitudinal passageway extending between the left element and the right element.

Clause 223—The system of clause 154, wherein the energy source includes radio-frequency (RF) energy.

Clause 224—The system of clause 154, wherein the energy source includes high-intensity focused ultrasound (HIFU).

Clause 225—The system of either one of clauses 223 or 224, wherein the energy source is configured to apply energy directly to the at least a first one of the plurality of chordae tendineae and the at least a second one of the plurality of chordae tendineae.

Clause 226—The system of either one of clauses 223 or 224, wherein the energy source is configured to apply energy to a fastener carried by the distal end of the shaft and configured to lock the at least a first one of the plurality of chordae tendineae and the at least a second one of the plurality of chordae tendineae with respect to each other.

Clause 227—The system of clause 226, wherein the energy source is configured to modify the fastener.

Clause 228—The system of clause 226, wherein the energy source is configured to at least partially melt the fastener.

Clause 229—The system of clause 226, wherein the energy source is configured to move the fastener from a first position to a second position.

Clause 230—The system of clause 226, wherein the energy source is configured to change the fastener from a first state to a second state.

Clause 231—The system of clause 230, wherein the fastener includes shape-memory alloy, and wherein the second state includes an Austenitic state.

Clause 232—The system of clause 226, wherein the energy source is configured to heat the fastener to cause the fastener to heat the at least a first one of the plurality of chordae tendineae and the at least a second one of the plurality of chordae tendineae.

Clause 233—A system includes a first catheter including an elongate shaft having a distal end and a proximal end and configured to be advanced into a ventricle of a heart of a subject; a grasper carried by the distal end of the shaft of the first catheter and configured to engage at least a first one of a plurality of chordae tendineae extending from a papillary muscle within the ventricle and attached to a first leaflet of an atrioventricular valve located between the ventricle and an adjacent atrium, and to engage at least a second one of the plurality of chordae tendineae attached to a second leaflet of the atrioventricular valve, and to further maintain simultaneous engagement of both of the at least a first one of the plurality of chordae tendineae and the at least a second one of the plurality of chordae tendineae; an actuator carried by the shaft of the first catheter and configured to cause the at least a first one of the plurality of chordae tendineae and the at least a second one of the plurality of chordae tendineae to be moved toward each other; a second catheter including an elongate shaft having a distal end and a proximal end and configured to be advanced into the ventricle; and a fastener carried by the distal end of the shaft of the second catheter and configured to lock the at least a first one of the plurality of chordae tendineae and the at least a second one of the plurality of chordae tendineae with respect to each other.

Clause 234—A system includes a catheter including an elongate shaft having a distal end and a proximal end and configured to be advanced into a ventricle of a heart of a subject; a grasper carried by the distal end of the shaft of the first catheter and configured to engage at least a first one of a plurality of chordae tendineae extending from a papillary muscle within the ventricle and attached to a first leaflet of an atrioventricular valve located between the ventricle and an adjacent atrium, and to engage at least a second one of the plurality of chordae tendineae attached to a second leaflet of the atrioventricular valve, and to further maintain simultaneous engagement of both of the at least a first one of the plurality of chordae tendineae and the at least a second one of the plurality of chordae tendineae; an actuator carried by the shaft of the first catheter and configured to cause the at least a first one of the plurality of chordae tendineae and the at least a second one of the plurality of chordae tendineae to be moved toward each other; a second catheter including an elongate shaft having a distal end and a proximal end and configured to be advanced into the ventricle; and an energy source carried by the distal end of the shaft of the second catheter and configured to deliver energy to fix the at least a first one of the plurality of chordae tendineae and the at least a second one of the plurality of chordae tendineae in place relative to each other.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

1. A method for treating a patient, comprising:
delivering a distal portion of an elongate medical device into a ventricle of a beating closed heart of a subject via a primary skin opening and a secondary puncture;
utilizing the distal portion of the elongate medical device to engage a plurality of chordae tendineae extending from a first papillary muscle within the ventricle, at least a first one of the plurality of chordae tendineae attached to a first leaflet of an atrioventricular valve located between the ventricle and an adjacent atrium, and at least a second one of the plurality of chordae tendineae attached to a second leaflet of the atrioventricular valve;
approximating the engaged plurality of chordae tendineae to increase an amount of closure possible between the first leaflet and the second leaflet; and
locking the plurality of chordae tendineae in their approximated condition.

2. The method of claim 1, wherein the atrioventricular valve comprises a mitral valve and the ventricle comprises a left ventricle.

3. The method of claim 1, wherein the atrioventricular valve comprises a tricuspid valve and the ventricle comprises a right ventricle.

4. The method of claim 3, wherein the first leaflet comprises an anterior leaflet, and wherein the second leaflet comprises a posterior leaflet.

5. The method of claim 3, wherein the first leaflet comprises a posterior leaflet, and wherein the second leaflet comprises a septal leaflet.

6. The method of claim 3, wherein the first leaflet comprises an anterior leaflet, and wherein the second leaflet comprises a septal leaflet.

7. (canceled)

8. The method of claim 1, wherein the ventricle comprises a left ventricle, and the secondary puncture comprises a puncture of an artery.

9. (canceled)

10. The method of claim 1, wherein the ventricle comprises a left ventricle, and wherein the secondary puncture comprises a puncture of a vein.

11. (canceled)

12. The method of claim 10, wherein delivering a distal portion of an elongate medical device into a ventricle comprises delivering the distal portion through a tertiary puncture in a septal wall between a right artium and a left atrium.

13. The method of claim 1, wherein the ventricle comprises a right ventricle, and wherein the secondary puncture comprises a puncture of a vein.

14-16. (canceled)

17. The method of claim 1, wherein the ventricle comprises a left ventricle, and wherein the secondary puncture comprises a puncture in an apex of the heart.

18-21. (canceled)

22. The method of claim 1, wherein the first papillary muscle comprises an anterolateral papillary muscle.

23. The method of claim 1, wherein the first papillary muscle comprises a posteromedial papillary muscle.

24-40. (canceled)

41. A method for treating a patient, comprising:
non-surgically engaging a plurality of chordae tendineae extending from a first papillary muscle within a ventricle, at least a first one of the plurality of chordae tendineae attached to a first leaflet of an atrioventricular valve located between the ventricle and an adjacent atrium, and at least a second one of the plurality of chordae tendineae attached to a second leaflet of the atrioventricular valve;
non-surgically approximating the engaged plurality of chordae tendineae to increase an amount of closure possible between the first leaflet and the second leaflet; and
non-surgically locking the plurality of chordae tendineae in their approximated condition.

42. The method of claim 41, wherein non-surgically engaging is performed with a first elongate medical device.

43. The method of claim 42, wherein non-surgically approximating is performed with the first elongate medical device.

44. The method of claim 42, wherein non-surgically approximating is performed with a second elongate medical device.

45. The method of claim 44, wherein non-surgically locking is performed with a third elongate medical device.

46. The method of claim 44, wherein non-surgically locking is performed with the second elongate medical device.

47-55. (canceled)

56. The method of claim 44, wherein the ventricle comprises a right ventricle, and wherein the first elongate medical device and the second elongate medical device are both inserted through a delivery lumen of an elongate delivery device.

57-234. (canceled)

235. The method of claim 1, further comprising:
engaging a second plurality of chordae tendineae extending from a second papillary muscle within the ventricle, at least a first one of the second plurality of chordae tendineae attached to the first leaflet of the atrioventricular valve, and at least a second one of the second plurality of chordae tendineae attached to the second leaflet of the atrioventricular valve;
approximating the engaged second plurality of chordae tendineae to further increase the amount of closure possible between the first leaflet and the second leaflet; and
locking the second plurality of chordae tendineae in their approximated condition.

236. The method of claim 1, wherein the primary skin opening comprises a puncture in the skin.

237. (canceled)

238. The method of claim 1, wherein the approximating proceeds substantially along an anterior-posterior axis.

239. The method of claim 1, wherein engaging comprises encircling.

240. The method of claim 1, wherein engaging comprises grasping with a grasper.

241. The method of claim 1, wherein approximating comprises reducing a distance between the at least a first one of the plurality of chordae tendineae and the at least a second one of the plurality of chordae tendineae.

242. The method of claim 241, wherein moving the at least a first one of the plurality of chordae tendineae and the at least a second one of the plurality of chordae tendineae toward each other comprises causing the at least a first one of the plurality of chordae tendineae and the at least a second one of the plurality of chordae tendineae to contact each other.

243. The method of claim 242, wherein locking comprises placing a constricting band around the at least a first one of the plurality of chordae tendineae and the at least a second one of the plurality of chordae tendineae.

244. The method of claim 1, wherein locking comprises placing a constrictor around the at least a first one of the plurality of chordae tendineae and the at least a second one of the plurality of chordae tendineae.

245. The method of claim 1, wherein locking comprises placing a clip around the at least a first one of the plurality of chordae tendineae and the at least a second one of the plurality of chordae tendineae.

246. The method of claim 42, wherein non-surgically locking is performed with an elongate locking device.
